(19) United States
(12) Patent Application Publication
(10) Pub. No.: US 2016/0022290 A1
(43) Pub. Date: Jan. 28, 2016

(54) RETRIEVAL AND CENTERING DEVICE AND METHOD WITH PRESSURE AND ULTRASOUND FEATURES

(71) Applicants: Eric Johnson, San Diego, CA (US); Joseph Lauinger, San Diego, CA (US); Jeremy Stigall, San Diego, CA (US); Gilbert Laroya, San Diego, CA (US); Paul Do, San Diego, CA (US)

(72) Inventors: Eric Johnson, San Diego, CA (US); Joseph Lauinger, San Diego, CA (US); Jeremy Stigall, San Diego, CA (US); Gilbert Laroya, San Diego, CA (US); Paul Do, San Diego, CA (US)

(21) Appl. No.: 14/774,735

(22) PCT Filed: Mar. 17, 2014
(86) PCT No.: PCT/US2014/030392

§ 371 Date: Sep. 11, 2015

Related U.S. Application Data
(60) Provisional application No. 61/794,016, filed on Mar. 15, 2013.

(51) Int. Cl. A61B 17/221; A61B 17/00

(52) U.S. Cl. CPC A61B 17/221; A61B 17/00234; A61B 2017/2212; A61B 2017/00336

(57) ABSTRACT

The present invention relates generally to devices and methods for retrieving or manipulating objects within a lumen. More specifically, embodiments of the invention relate to devices and methods for retrieving or manipulating medical devices from a body lumen. One embodiment of the present invention provides a novel and improved retrieval snare and method of fabricating and using the same. The snare includes a snare wire, having a distal end and a proximal end, for use in the human anatomy, such as but not limited to blood vessels, pulmonary airways, reproductive anatomy, gastrointestinal anatomy, and organs such as the kidneys or lungs. The device enables a user to capture a foreign object located within the human anatomy, grasp said object in a controlled manner, and retrieve and remove said object from the human anatomy.

### CROSS REFERENCE TO RELATED APPLICATIONS

This application claims priority to U.S. provisional application No. 61/794,016 filed Mar. 15, 2013, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

The following patents and patent applications are herein incorporated by reference in their entirety: U.S. patent application Ser. No. 11/969,827 titled, “ENDOLUMINAL FILTER WITH FIXATION” filed on Jan. 4, 2009.

### FIELD

Embodiments of the invention relate generally to devices and methods for retrieving or manipulating objects within a lumen. More specifically, embodiments of the invention relate to devices and methods for retrieving or manipulating medical devices from a body lumen.

### BACKGROUND

Embolic protection is utilized throughout the vasculature to prevent the potentially fatal passage of embolic material in the bloodstream to smaller vessels where it can obstruct blood flow. The dislodgement of embolic material is often associated with procedures which open blood vessels to restore natural blood flow such as stenting, angioplasty, arthrectomy, endarterectomy or thrombectomy. Used as an adjunct to these procedures, embolic protection devices trap debris and provide a means for removal for the body.

One widely used embolic protection application is the placement of filtration means in the vena cava. Vena cava filters (VCF) prevent the passage of thrombus from the deep veins of the legs into the blood stream and ultimately to the lungs. This condition is known as deep vein thrombosis (DVT), which can cause a potentially fatal condition known as pulmonary embolism (PE).

The next advancement in filters added the element of recoverability. Retrievable filters were designed to allow removal from the patient subsequent to initial placement. These filters can incorporate retrieval features that can be grasped and/or secured by a retrieval device, such as a snare based retrieval device. Grasping the retrieval feature using a snare generally requires the user to manipulate the snare over the retrieval feature, which can be difficult due to a variety of factors, such as retrieval feature geometry and location within the lumen, the structure and properties of the snare, and ability to visualize the retrieval feature and/or snare using a real-time visualization technique such as fluoroscopy.

Accordingly, it would be desirable to have an improved retrieval device that would facilitate engagement with a retrieval feature on a device making retrieval and/or manipulation of the device easier and faster to complete.

### SUMMARY OF THE DISCLOSURE

The present invention relates generally to devices and methods for retrieving or manipulating objects within a lumen. More specifically, embodiments of the invention relate to devices and methods for retrieving or manipulating medical devices from a body lumen.

One embodiment of the present invention provides a novel and improved retrieval snare and method of fabricating and using the same. The snare includes a snare wire, having a distal end and a proximal end, for use in the human anatomy, such as but not limited to blood vessels, pulmonary airways, reproductive anatomy, gastrointestinal anatomy, and organs such as the bladder, kidneys or lungs. The device enables a user to capture a foreign object located within the human anatomy, grasp said object in a controlled manner, and retrieve and remove said object from the human anatomy. Examples of foreign objects which might be removed from the human anatomy include implants such as stents, guidewires, leads, sheaths, filters, and valves, and organic objects such as kidney stones or calcified emboli. Other areas where embodiments of the snare can be used include, for example, removal and/or repositioning of distal protection devices that are used in a variety of medical procedures such as carotid stenting and percutaneous aortic valve replacement; and abdominal aortic aneurysm and thoracic aortic aneurysm devices. For example, a snare can be used to capture a vena cava filter and pull it into a retrieval sheath for removal from the patient. The snare is advanced through one or more retrieval sheaths, up to the site of a deployed filter. The snare is then deployed into the vessel, and engaged with the filter. Finally, the snare is held under tension while the sheath is advanced over said filter, collapsing it into the ID of said sheath. Another example is the use of a snare to grasp and extract loose kidney stones from a patient's kidneys. The snare is advanced through one or more sheaths, up to the site of the loose kidney stone. The snare is then deployed and engaged with the stone. Next, the snare is pulled into the sheath, drawing the stone into the distal ID of said sheath.

In some embodiments, a device for retrieving an object from a lumen is provided. The device includes a sheath configured to fit within the lumen, the sheath having a proximal end and a distal end. A snare can be disposed within the sheath. The snare can have a shaft with a longitudinal axis, a proximal end and a distal end and a plurality of loop elements in connection with the distal end of the shaft. The plurality of loop elements can have a collapsed configuration within the sheath and at least one deployed configuration outside the sheath. The plurality of loop elements can be configured to be deployed through an opening at the distal end of the sheath. The at least one deployed configuration can include a fully deployed configuration in which the plurality of loop elements are deployed in a propeller-like configuration.

In some embodiments, the first sheath includes a flexible distal tip portion that is configured to invert when the object is withdrawn into the sheath.

In some embodiments, a plurality of sheaths includes flexible distal tip portions that are configured to invert when the object is withdrawn into the sheaths.

In some embodiments, the plurality of loop elements in the fully deployed configuration are angled less than 90 degrees with respect to the longitudinal axis of the shaft such that the plurality of loop elements has an axial reach both proximal and distal the distal end of the shaft.

In some embodiments, each of the plurality of loop elements includes at least one shape memory wire and one radiopaque wire.

In some embodiments, the shape memory wire is made of a nickel titanium alloy and the radiopaque wire is made of platinum.

In some embodiments, the loop elements in the fully deployed configuration are arranged to form a circle geometry when viewed along the longitudinal axis.

In some embodiments, the object being retrieved by the device is a filter having a retrieval element and a support member, and wherein the axial reach of the loop elements in the fully deployed configuration is less than the distance between the retrieval element and the support member.

In some embodiments, the proximal portion of the sheath and the proximal portion of the shaft are connected with a snap fitting.

In some embodiments, the proximal portion of the outer sheath and the proximal portion of the inner sheath are connected with a snap fitting.

In some embodiments, the device further includes an outer sheath, wherein the sheath is disposed within the outer sheath.

In some embodiments, the outer sheath has greater column strength than the inner sheath.

In some embodiments, the loop elements have a plurality of deployment configurations, and wherein the proximal portion of the shaft includes a plurality of indicators that correspond to the plurality of deployment configurations.

In some embodiments, the plurality of indicators includes a plurality of detents.

In some embodiments, the proximal portion of the sheath includes a first tactile identifier and the proximal portion of the shaft includes a second tactile identifier, wherein the first tactile identifier is different from the second tactile identifier.

In some embodiments, the at least one deployed configuration includes an initial deployed configuration in which the plurality of loop elements are deployed substantially transversely with respect to the longitudinal axis.

In some embodiments, the plurality of loop elements is deployed in a clover leaf configuration in the initial deployed configuration.

In some embodiments, the at least one deployed configuration includes an intermediate deployed configuration in which the plurality of loop elements are deployed substantially axially with respect to the longitudinal axis.

In some embodiments, a method for capturing an object in a lumen defined by a lumen wall is provided. The method includes advancing a sheath within the lumen, the sheath having a proximal end and a distal end, until the distal end of the sheath is proximal the object; deploying a plurality of loop elements of a snare out of the distal end of the sheath in a propeller-like configuration; and capturing a portion of the object with at least one of the plurality of loop elements.

In some embodiments, the method further includes withdrawing the loop elements in a proximal direction to engage the portion of the object.

In some embodiments, the method further includes rotating the loop elements to engage the portion of the object.

In some embodiments, the method further includes retracting the portion of the object within the sheath.

In some embodiments, the method further includes advancing an outer sheath over the object.

In some embodiments, the method further includes advancing the snare to a full deployment detent on the snare.

In some embodiments, the method further includes visualizing the snare in the lumen using fluoroscopy.

In some embodiments, the method further includes decoupling a snap fitting holding together the sheath and the snare.

In some embodiments, the method further includes decoupling a snap fitting holding together the outer sheath and the inner sheath.

In some embodiments, a device for retrieving an object from a lumen is provided. The device can include a sheath configured to fit within the lumen, the sheath having a proximal end, a distal end and a radiopaque marker offset from the distal end. A snare can be disposed within the sheath, the snare having a shaft with a longitudinal axis, a proximal end and a distal end and a plurality of loop elements in connection with the distal end of the shaft. The plurality of loop elements can have a collapsed configuration within the sheath and at least one deployed configuration outside the sheath. The plurality of loop elements can be configured to be deployed through an opening at the distal end of the sheath. At least one deployed configuration can include an initial deployed configuration in which the plurality of loop elements is deployed substantially transversely with respect to the longitudinal axis.

In some embodiments, the plurality of loop elements are deployed in a clover leaf configuration in the initial deployed configuration.

In some embodiments, the plurality of loop elements are deployed in an elliptical or oblong configuration in the fully deployed configuration.

In some embodiments, the at least one deployed configuration includes a fully deployed configuration in which the plurality of loop elements are deployed in substantially circular configuration.

In some embodiments, the radiopaque marker is offset about 3 to 5 mm from the distal end of the sheath.

In some embodiments, a specific radiopaque marker pattern is disposed on each of the loop elements to enable visual differentiation of each loop element fluoroscopically. For example, each loop element can have a different number of radiopaque markers.

In some embodiments, a method for capturing an object in a lumen defined by a lumen wall is provided. The method includes advancing a sheath within the lumen, the sheath having a proximal end and a distal end, until the distal end of the sheath is proximal the object; deploying a plurality of loop elements of a snare out of the distal end of the sheath until the loop elements achieve substantially full apposition with the circumference of the lumen wall; and capturing a portion of the object with at least one of the plurality of loop elements.

In some embodiments, the method further includes aligning a radiopaque marker offset from the distal end of the sheath with a radiopaque feature of the object.

In some embodiments, the radiopaque feature of the object is a retrieval element.

In some embodiments, a device for retrieving an object from a lumen defined by a lumen wall is provided. The device can include a sheath configured to fit within the lumen, the sheath having a proximal end and a distal end; and a snare slidably disposed within the sheath, the snare having a shaft with a longitudinal axis, a proximal end and a distal end and a plurality of loop elements in connection with the distal end of the shaft, wherein each of the plurality of loop element has a proximal portion and a distal portion, wherein the plurality of loop elements has a collapsed configuration within the sheath and at least one deployed configuration outside the sheath, wherein the plurality of loop elements are configured to be deployed through an opening at the distal end of the sheath, wherein the at least one deployed configuration includes a fully deployed configuration in which the plurality of loop elements are deployed such that the distal portions of the loop elements are arranged in a substantially continuous, circumferential, planar and oblong configuration that is transverse to the longitudinal axis.

In some embodiments, the sheath includes a flexible distal tip portion that is configured to invert when the object is withdrawn into the sheath.

In some embodiments, the plurality of loop elements in the fully deployed configuration are angled less than 90 degrees with respect to the longitudinal axis of the shaft such that the plurality of loop elements has an axial reach both proximal and distal the distal end of the shaft.

In some embodiments, each of the plurality of loop elements includes at least one shape memory wire and one radiopaque wire. In some embodiments, the shape memory wire is made of a nickel titanium alloy and the radiopaque wire is made of platinum.

In some embodiments, the proximal portions of the plurality of loop elements comprise spoke portions that are secured together with a flexible sleeve.

In some embodiments, the object is a filter having a retrieval element and a support member, and wherein the axial reach of the loop elements in the fully deployed configuration is less than the distance between the retrieval element and the support member.

In some embodiments, the proximal portion of the sheath and the proximal portion of the shaft are connected with a snap fitting.

In some embodiments, the device further includes an outer sheath, wherein the sheath is disposed within the outer sheath.

In some embodiments, the outer sheath has greater column strength than the sheath.

In some embodiments, the loop elements have a plurality of deployment configurations, and wherein the proximal portion of the shaft includes a plurality of indicators that correspond to the plurality of deployment configurations. In some embodiments, the plurality of indicators comprise a plurality of detents. In some embodiments, the proximal portion of the sheath includes a first tactile identifier and the proximal portion of the shaft includes a second tactile identifier, wherein the first tactile identifier is different from the second tactile identifier.

In some embodiments, the at least one deployed configuration includes an initial deployed configuration in which the plurality of loop elements are deployed substantially axially with respect to the longitudinal axis.

In some embodiments, the distal portions of the plurality of loop elements in the fully deployed configuration are configured to achieve complete circumferential apposition with the lumen wall. In some embodiments, the lumen wall can define a lumen that is oblong or circular or that changes between oblong and circular.

In some embodiments, the at least one deployed configuration includes an intermediate deployed configuration in which the plurality of loop elements are deployed substantially transversely with respect to the longitudinal axis.

In some embodiments, a device for retrieving an object from a lumen is provided. The device can include a sheath configured to fit within the lumen, the sheath having a proximal end, a distal end and a radiopaque marker offset from the distal end; and a snare disposed within the sheath, the snare having a shaft with a longitudinal axis, a proximal end and a distal end and a plurality of loop elements in connection with the distal end of the shaft, wherein the plurality of loop elements has a collapsed configuration within the sheath and at least one deployed configuration outside the sheath, wherein the plurality of loop elements are configured to be deployed through an opening at the distal end of the sheath, wherein the at least one deployed configuration includes an initial deployed configuration in which the plurality of loop elements are deployed substantially transversely with respect to the longitudinal axis.

In some embodiments, the at least one deployed configuration includes a fully deployed configuration in which the plurality of loop elements are deployed in substantially circular configuration.

In some embodiments, the radiopaque marker is offset about 3 to 5 mm from the distal end of the sheath.

In some embodiments, the at least one deployed configuration includes a fully deployed configuration in which the plurality of loop elements are deployed in substantially oblong configuration.

In some embodiments, the plurality of loop elements each includes a loop collapse facilitator.

In some embodiments, the plurality of loop elements are secured together with sleeves.

In some embodiments, a method for capturing an object in a lumen defined by a lumen wall is provided. The method can include advancing a sheath within the lumen, the sheath having a proximal end and a distal end, until the distal end of the sheath is proximal the object; deploying a plurality of loop elements of a snare out of the distal end of the sheath until the loop elements achieve substantially full apposition with the circumference of the lumen wall; and capturing a portion of the object proximate to the lumen wall with at least one of the plurality of loop elements.

In some embodiments, the method further includes aligning a radiopaque marker offset from the distal end of the sheath with a radiopaque feature of the object.

In some embodiments, the radiopaque feature of the object is a retrieval element.

In some embodiments, the method further includes advancing the distal end of the sheath over the captured object.

In some embodiments, the distal end of the sheath inverts as the sheath is advanced over the captured object.

In some embodiments, a method for capturing an object in a lumen defined by a lumen wall is provided. The method includes advancing a sheath within the lumen, the sheath having a proximal end and a distal end, until the distal end of the sheath is proximal the object; determining the position of the object within the lumen; deploying a plurality of loop elements of a snare out of the distal end of the sheath to one of a plurality of predetermined loop element deployment configurations based on the determination of the position of the object; and capturing a portion of the object with at least one of the plurality of loop elements.

In some embodiments, the plurality of loop elements are deployed to the predetermined loop element deployment configuration using a deployment indicator.

In some embodiments, the method further includes advancing an inner sheath disposed with the sheath over a portion of the object and advancing the sheath over the entire object.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is an axial view of the distal end of one embodiment of the snare device, showing the loop elements which substantially form a complete circle about the axis of the shaft. The edges of each loop overlap adjacent loops to ensure a substantially continuous circular pattern.

FIG. 1B is a side perspective view of the snare device shown in FIG. 1A, showing the loop elements such that the plurality of loop elements has an axial reach both proximal and distal the distal end of the shaft.

FIG. 1C is a side cross-sectional view of a stowed snare within both an outer sheath and an inner sheath.

FIGS. 1D-IF illustrate the various deployment stages of the loop elements of one embodiment of the snare. FIGS. 1D and 1E illustrate an initial deployment stage of the loop elements, while FIG. 1F illustrates an intermediate deployment stage of the loop elements.

FIGS. 1G and 1H illustrate the flexible distal tip portion of the sheath with a deployed snare (FIG. 1G) and a partially stowed snare (FIG. 1H).

FIGS. 1I-1J illustrate snare embodiments having two loop elements with a substantially elliptical or oblong fully deployed configuration.

FIGS. 1K-1M illustrate snare embodiments having two loop elements with a substantially elliptical or oblong fully deployed configuration and a loop collapse facilitator.

FIGS. 1N-1Q illustrate the stages of deployment of an embodiment of a snare with two loop elements.

FIG. 1R illustrates a snare embodiment having two loop elements with a substantially elliptical or oblong fully deployed configuration, and a plurality of radiopaque markers disposed on each loop in different patterns, to differentiate each loop element fluoroscopically.

FIG. 1S is a side view of a snare embodiment having two loop elements with a substantially elliptical or oblong fully deployed configuration, showing the loop elements having both a distal and proximal reach.

FIG. 1T illustrates a snare embodiment having four loop elements in a substantially circular fully deployed configuration, and a plurality of radiopaque markers disposed on each loop in different patterns, to differentiate each loop element fluoroscopically.

FIG. 1U illustrates another snare embodiment having two loop elements with a substantially elliptical or oblong fully deployed configuration and a loop collapse facilitator.

FIGS. 1V-1X illustrate another snare embodiment having two loop elements that are fastened together at the swage and attached together with sleeves.

FIG. 2A is an end view of an embodiment of a single loop element, using a single nitinol wire wrapped with a single radiopaque platinum wire.

FIG. 2B is a perspective view of the single loop element shown in FIG. 2A.

FIG. 3A is a side view of another embodiment of a single loop on the end of a snare device, to illustrate the relative geometry of the loop elements.

FIG. 3B is an end view of the single loop shown in FIG. 3A.

FIG. 4 is an end view of a loop element and a hypo tube, to illustrate the D shape or pie shape geometry of the loop element features.

FIG. 5A is an end view of an embodiment of a single loop element, using a plurality of wires which are twisted together to form a strand.

FIG. 5B is a close up view of a portion of the single loop element strand shown in FIG. 5A.

FIG. 6A illustrates an embodiment of a single loop element, using a plurality of wires which are braided together to form a strand.

FIG. 6B illustrates a close up view of a portion of the single loop element strand shown in FIG. 6A.

FIG. 7 is a side view of an embodiment of a snare device using single wire loop elements, and a steel hypo tube which attaches the loops to the shaft via a crimp process.

FIG. 8 is a close up view of the snare device shown in FIG. 7, further illustrating the steel hypo tube which attaches the loops to the shaft via a crimp process.

FIG. 9 is a perspective view of the snare device shown in FIG. 7.

FIG. 10 is an end view of the snare device shown in FIG. 7. The view illustrates how the loops overlap laterally, with the outer perimeter forming a circular shape.

FIG. 10A is an end view of another embodiment of a snare device. The view illustrates how the loop elements are twisted together laterally, with the outer perimeter forming a circular shape.

FIG. 11 is a side view of an embodiment of a snare assembly, where the loop elements are attached to the shaft element with a wire coil.

FIG. 12 is a side view of an embodiment of the shaft, hypo tube, and a single loop element for illustrative purposes. The actual snare device can have a plurality of loop elements. The view illustrates an embodiment of the loop element wherein the angle of the radius portion of the loop element is typically about 45 degrees from the central axis of the hypo tube component.

FIG. 13 is a side view of an alternate embodiment of the snare device where the shaft is made from a twisted strand, and the loop elements form a circular shape in a single plane 90 degrees from the axis of the shaft.

FIG. 14 is a horizontal isometric view of the alternate embodiment shown in FIG. 13, illustrating the flat circular shape of the outer perimeter of the snare loops.

FIG. 15 is a frontal angled view of the alternate embodiment shown in FIG. 13, illustrating the circular shape of the snare outer perimeter, as well as the straight portions of each loop overlapping the adjacent loop to form a closed circle with no gaps about the perimeter.

FIGS. 16-19 illustrate embodiments of methods of using any of the snares 10 disclosed herein.

FIGS. 20-22 illustrate embodiments of a snap fitting that can be used with the snare.

FIGS. 23A-23C illustrate an embodiment of guidewire having both a pressure sensor and an IVUS transducer.

FIGS. 24A-24D illustrate two embodiments of an intravascular ultrasound catheter joined together in parallel with a catheter.

FIGS. 25A and 25B illustrate an embodiment of a filter delivery system where the pressure sensor and/or IVUS transducer are integrated into a delivery catheter, a retrieval catheter or a device itself.

FIGS. 26A-26G illustrate various embodiments of a retrieval system having an ultrasound transducer incorporated into a sheath or a snare.

FIGS. 27A-27C illustrate various embodiments of a centering device that positions an ultrasound transducer in the center of a lumen, or alternatively, places an array of ultrasound transducers around the periphery of the lumen.

FIG. 28 illustrates a method of using a retrieval system having one or more ultrasound transducers to retrieve a filter from a body lumen.

FIG. 29 is a section view of a wire strut or support element of a filter (w/s/s) having multiple segments in a concentric arrangement.

FIG. 30 is an embodiment of a segment having one or a plurality of laser drilled holes formed therein.

FIG. 31 is an embodiment of a segment having one or a plurality of raised features or alternatively roughed portions formed thereon.

FIG. 32 is an embodiment of a segment having one or a plurality of bubbles formed therein.

FIG. 33 is an embodiment of a segment having one or a plurality of dimples formed therein.

FIG. 34 is an embodiment of a segment having a coil or braided structure within or about the segment.

FIG. 35 is an embodiment of a segment having a plurality of echogenic markers arrayed in rings about the segment to provide an indication of measurement via the spacing between adjacent rings.

FIG. 36 illustrates various alternative configurations for a segment used alone or in conjunction with other segments.

FIG. 37 is a view of an exemplary filter illustrating various alternative aspects of providing a filter with improved echogenic characteristics.

### DETAILED DESCRIPTION

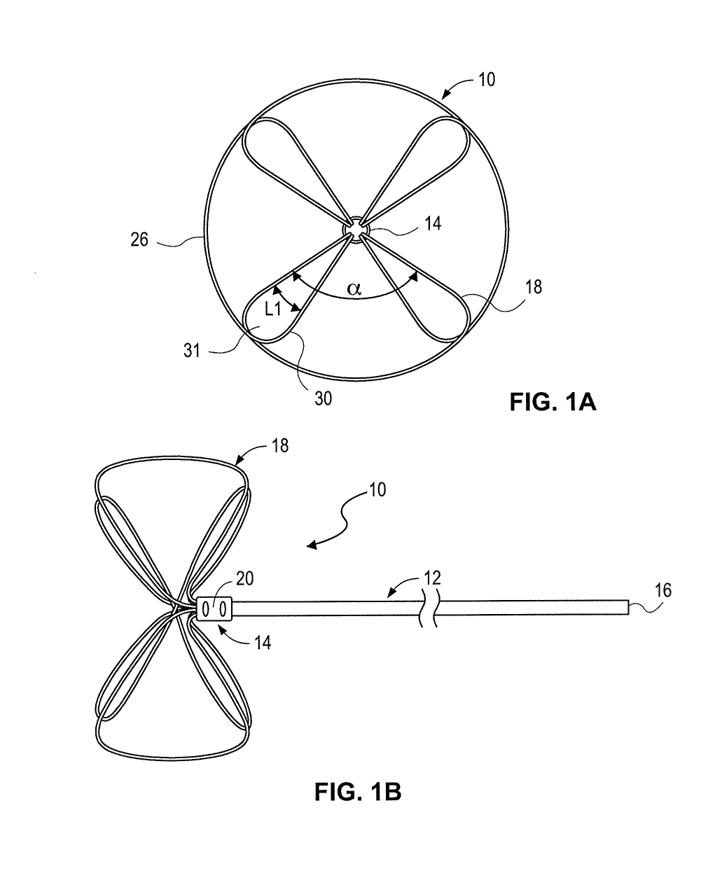

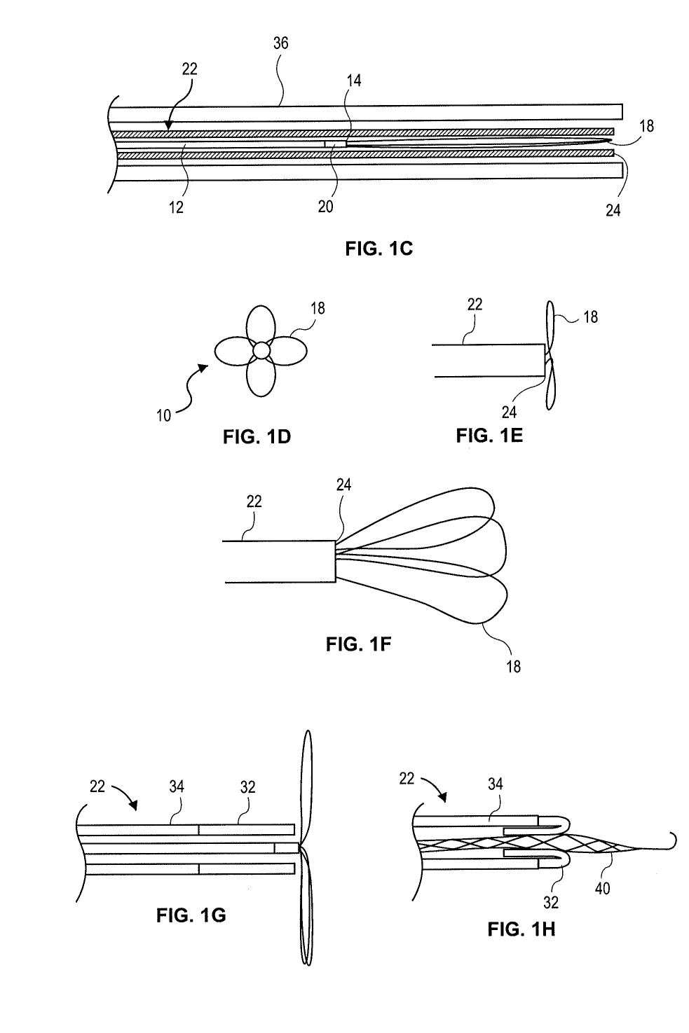

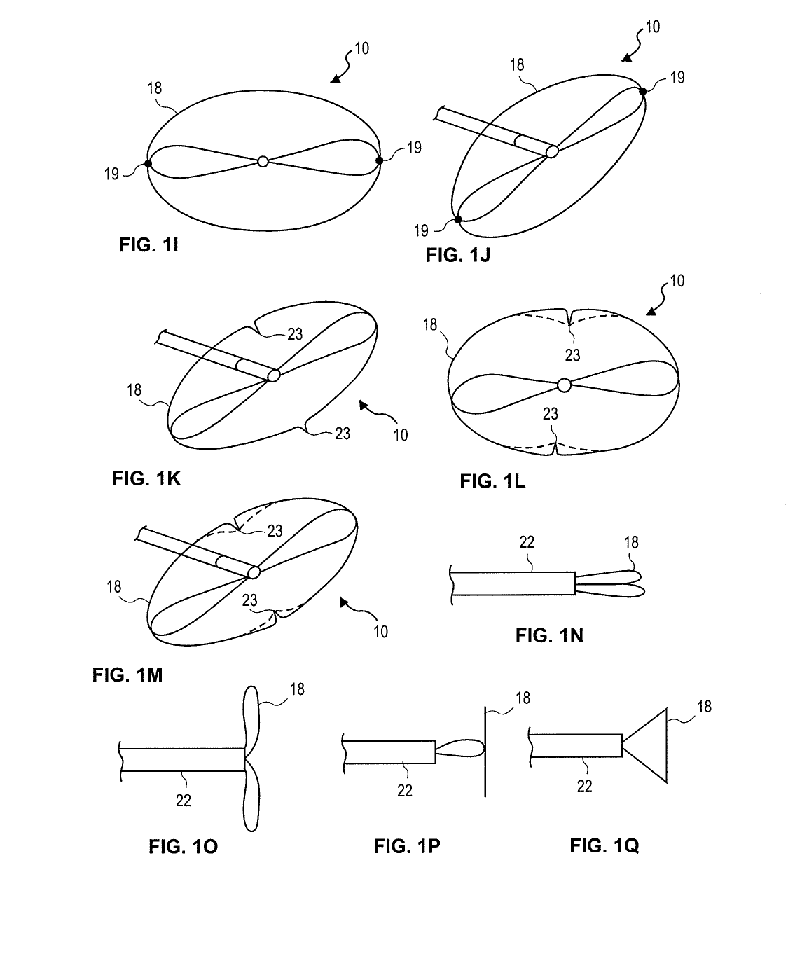

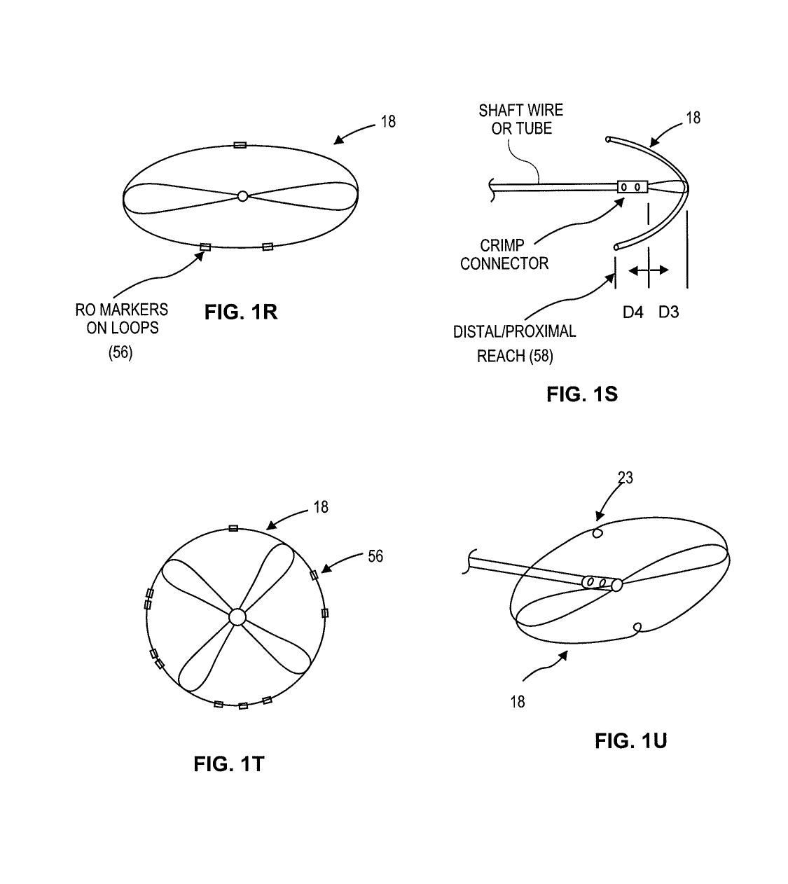

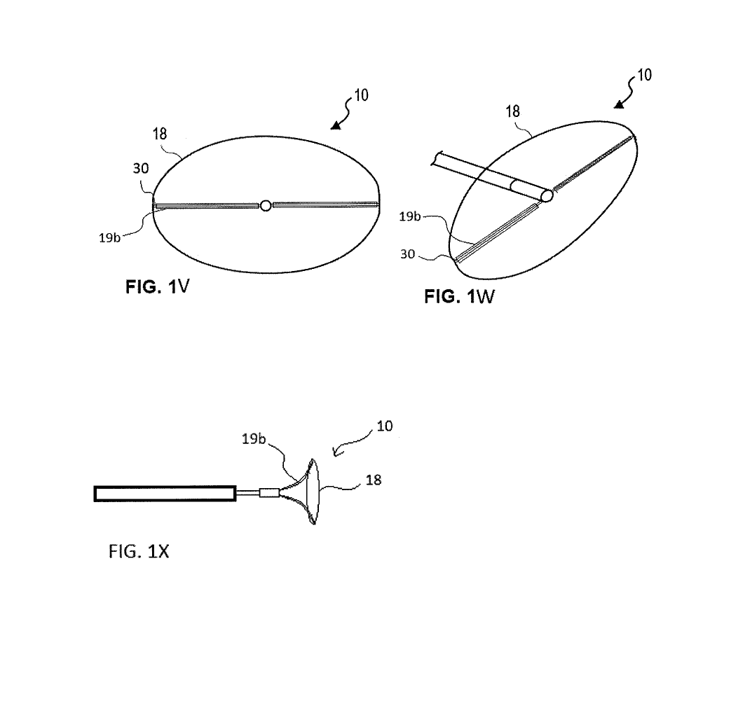

As illustrated in FIGS. 1A and 1B, an embodiment of a retrieval device 10, such as a snare, includes a primary or main shaft 12, having a distal end 14 and a proximal end 16. At the distal end 14 of the shaft 12 is a plurality of loop elements 18. In some embodiments, the device 10 can typically have at least two loop elements 18, but can have three or more loop elements 18. These loop elements 18 are attached proximally to the distal end 14 of the shaft 12 via a hypo tube component 20, and can be free and independent at their distal-most ends. In other embodiments, the distal ends of the loop elements 18 can be fastened or connected to adjacent loop elements using, for example, loop connectors, as described in more detail below. The loops 18 can be of a polymeric or metallic material, and are typically radiopaque and flexible.

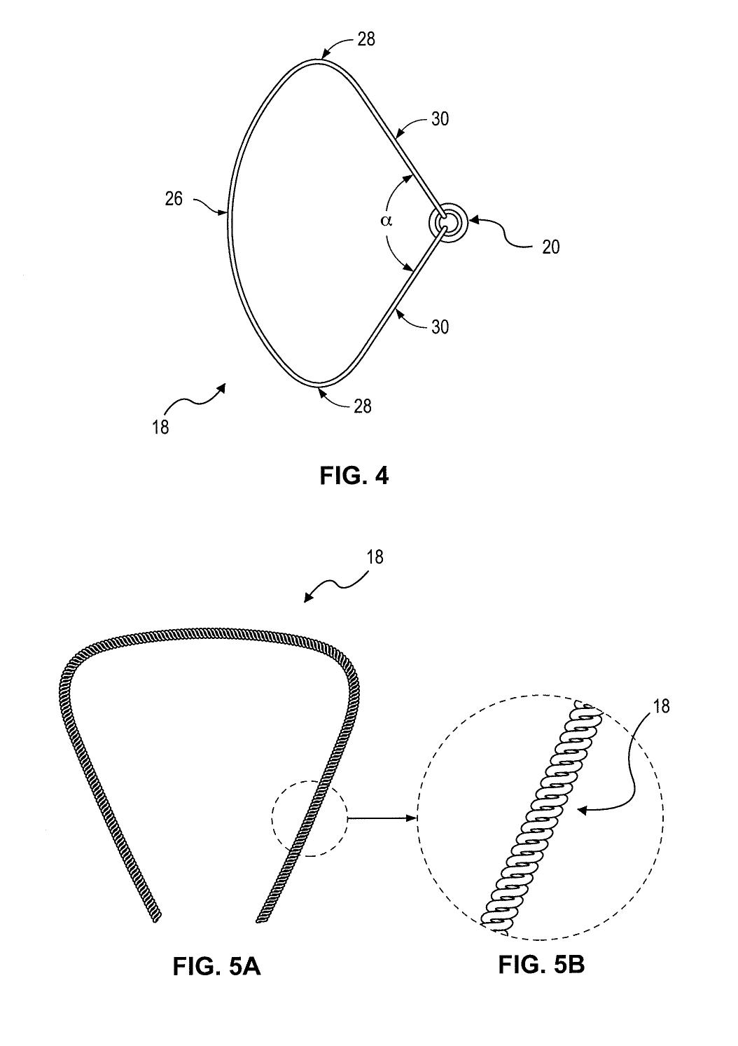

The loop elements 18 can have a region of overlap 31, with a span L1, between the adjacent loop elements. In some embodiments, L1 can be less than about 5, 10, 15, 20, 25, 30, 35, 40 or 45 degrees. In some embodiments, L1 can be between about 0 to 45 degrees, or about 0 to 15 degrees. The span of radial or circumferential coverage by each loop element 18 can be defined by the angle α between the two spoke elements 30 of the loop element 18, as shown in FIG. 1A and FIG. 4. In some embodiments, angle α depends on the number of loop elements 18 and the amount of loop element overlap, L1. For example, in some embodiments, angle α can be determined approximately by dividing 360 degrees by the number of loop elements and then adding the amount of overlap. L1. Thus, for a four loop element snare embodiment with 10 degrees of overlap between each loop element, angle α equals approximately 100 degrees. For a two loop element snare embodiment with 10 degrees of overlap, angle α equals about 190 degrees. In other embodiments, the radial or circumferential coverage of the loop elements can be different while still providing complete radial or circumferential coverage. For example, in a four loop element embodiment with 10 degrees overlap, two loop elements can have an angle α of about 130 degrees while the other two loop elements can have an angle α of about 70 degrees.

The shape and flexibility of the loop elements 18 allows them to collapse and/or fold down easily into, for example, a 7 Fr or smaller sheath catheter 22 during loading of the device 10 into the sheath 22 and/or during deployment of the device 10 from the sheath 22 and retraction of the device 10 into the sheath 22, as illustrated in FIG. 1C. In some embodiments, an additional outer sheath 36 can be used to provide additional column strength. In some embodiments, the outer sheath 36 can be a braided sheath, while the inner sheath 22 can be a coiled sheath, which can be more flexible that the braided sheath. The outer sheath 36 can be used with any of the embodiments disclosed herein.

In some embodiments, as illustrated in FIGS. 1G and 1H, the sheath 22, which can be used in a single sheath embodiment or as an inner sheath in a double sheath embodiment, can have a soft, flexible and elastic distal tip portion 32 that can expand over a foreign object, such as a filter 40, that is being pulled into the sheath 22. In addition, the flexible distal tip portion 32 can evert when the foreign object and/or deployed loop elements 18 are retracted back into the sheath 22. When the flexible distal tip portion 32 inverts, it can form a ramp-like structure that facilitates the retraction of the filter 40 and the loop elements 18 back into the sheath 22. The main portion 34 of the sheath 22 can have stiffer column strength than the flexible distal tip portion 32 in order to tolerate the relatively high levels of force that can be generated while pulling out embedded filters with the device 10. In some embodiments, as mentioned above, an outer sheath can be used to provide additional column strength if needed.

In some embodiments, the distal tip portion 32 of the sheath 22 can be radiopaque and/or include a radiopaque marker. For example, in some embodiments, the polymer forming the distal tip portion 32 can be doped with radiopaque elements or compounds, such as barium, tantalum, tungsten, palladium, platinum or iridium based compounds or elements. Alternatively or in addition to the radiopaque doping, a single or plurality of radiopaque markers, such as a radiopaque marker band made of the radiopaque elements or compounds described herein, can be incorporated into the distal tip portion 32. In some embodiments, the radiopaque marker band can be offset approximately 1-10 mm, or about 3-mm from the distal end of the sheath 22, so as to not interfere with the elasticity and eversion of the distal tip portion 32 during the capture process. The radiopaque doping and/or marker allow the operator to visualize the location of the distal tip portion 32 of the sheath 22 during insertion, advancement, and positioning of the sheath 22 near the foreign object within the lumen. This allows the operator to accurately and precisely advance and position the tip of sheath 22 to the foreign object. In some embodiments where an outer sheath is combined with the retrieval sheath, each sheath can employ different radiopaque marker patterns to allow the operator to differentiate between the two sheaths fluoroscopically.

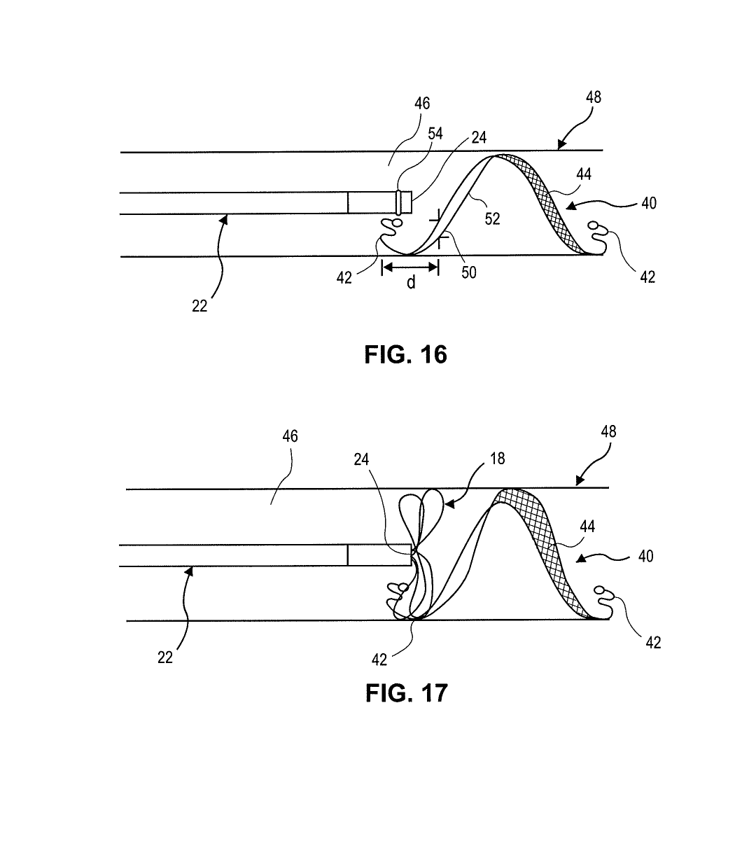

In addition, the marker offset can also function as an alignment feature which aids the operator in positioning the distal end of the sheath 22 in the proper location relative to the foreign object to be retrieved. For example, the foreign object can be a filter 40 with a frame 52, a plurality of anchors 50 on the frame 40 and a retrieval element 42 as illustrated in FIGS. 16-19. In some embodiments, deployment of the loop elements 18 is ideally distal the retrieval element 42 but proximal the anchor 50 closest to the retrieval element 42, which can be achieved be lining up the marker band 54 with an element or feature on the filter 40, such as the retrieval element 42, for example. The distance d between the retrieval element 42 and the anchor 50 can serve as a design constraint for loop element 18 deployments, where the loop elements 18 can be designed to deploy with an axial reach of less than the distance d between the retrieval element 42 and the anchor 50 or other feature on the filter 40. FIGS. 16-19 are more fully described below.

In some embodiments, the shaft 12 is straight and can be made of polymeric or metallic material, for example. The shaft 12 can be made of a solid design such as a wire, but can alternatively be hollow to facilitate passage of secondary devices through a lumen in the shaft 12. The shaft 12 can be of a single wire or element, but can also be constructed of a plurality of wires or elements which can be braided, twisted or stranded into a single shaft 12. The shaft 12 provides a means by which the user can advance, manipulate, and retract the distal end 14 of the device to capture and remove a foreign object from the human body. Typically, the user manipulates the device 10 at the proximal end 16, which is typically outside of the human anatomy. By manipulating the shaft 12, the motion is translated to the distal end 14 of the device 10, which in turn causes the loop elements 18 to move within the human anatomy. This motion allows the loop elements 18 to catch on the foreign object to be removed from the body. Consequently, the shaft 12 can be designed to have sufficient stiffness, flexibility, pushability and torqueability to accomplish the functions described herein. In some embodiments, a single wire shaft can provide sufficient stiffness, flexibility, pushability and torqueability. In other embodiments, a multiple wire shaft can provide sufficient stiffness, flexibility, pushability and torqueability.

In some embodiments, a hypo tube 20 attaches the loop elements 18 to the shaft 12. The hypo tube 20 has an inner diameter and an outer diameter, and is typically sized such that the shaft 12 and all of the loop elements 18 can fit within the inner diameter of the hypo tube 20. The inner diameter is sized such that there is adequate interference between the hypo tube 20 and the shaft 12 and the loop elements 18, so that the hypo tube 20 can be swaged or crimped circumferentially, mechanically locking the loop elements 18 and shaft 12 together. Additionally, the hypo tube can be radially shaped into a non-circular shape, such as but not limited to a hexagon or square or other rectilinear shape, to further facilitate mechanical fit and locking of said shaft 12 and loop elements 18. In some embodiments, the length of the hypo tube 20 is about at least two times its outer diameter, but can be as short as one times its outer diameter, or as long as twenty times its outer diameter. The loop elements 18 can also be attached to the shaft 12 via welding, soldering, capturing within a coil, or potting within a polymeric or rigid adhesive form, for example.

In some embodiments, the loop elements 18 have a geometric shape which allows them to deploy in a staged manner, where the shape and effective diameter of the snare 10 is dependent upon how far the snare 10 is deployed out of the sheath 22. In a first deployment stage as shown in FIG. 1D, the loops 18 are initially deployed from the sheath 22 and expand, each with a semi-circular shape, a semi-oval shape, or semi-oblong shape, for example, and the effective diameter of the snare 10 is smaller than the effective diameter when the snare 10 is fully deployed. In some embodiments such as a four loop elements 18 embodiment, the snare geometry in the first deployment stage resembles a cloverleaf shape. In some embodiments, as illustrated in FIG. 1E, the cloverleaf shaped loops 18 extend substantially transversely from the shaft 12 and sheath 22. In a second deployment stage as shown in FIG. 1F, the loops 18 extend further from the sheath 22. In some embodiments, in the second deployment stage the loops 18 extend both transversely and axially from the distal end 24 of the sheath 22, thereby providing the snare 10 with extended axial reach in this configuration. In a third deployment stage as illustrated in FIG. 1A, the loops 18 fully expand, reaching the full effective diameter of the snare 10. The snare 10 geometry in the third deployment stage can resemble a substantially complete circle, when viewed along the longitudinal axis of the snare 10 to yield an end view as shown in FIG. 1A, with spoke elements that lead from the circle towards the central hypo tube attachment point. The circle geometry created by the radial edge portions of the loop elements 18 eliminates or reduces gaps between the loop elements 18, which can make it easier for the operator to engage a retrieval element on a foreign object with the snare 10, especially when the retrieval element is located near or around the periphery of the lumen.

To facilitate engagement of the loop elements 18 with the retrieval element, the loop elements 18, when fully deployed, can be sized to conform approximately to the inner diameter of the lumen in which the foreign object is located. This allows full or substantially full apposition between the loop elements 18 and the full circumference of the lumen wall, which enhances the ability of the snare 10 to capture the retrieving element. In some embodiments, the geometry of the fully deployed loop elements 18 can be substantially elliptical, oval or oblong in order to conform to a lumen with a substantially elliptical, oval or oblong cross-sectional geometry. In these embodiments, the major axis of the elliptical or oblong geometry can be sized to conform approximately to the inner diameter of the lumen in which the foreign object is located. In general terms, the geometry of the fully deployed loop elements 18 can substantially match the geometry of the lumen.

For example, the vena cava may have a generally elliptical or oblong cross-sectional geometry. For use in the vena cava, a snare 10 with loop elements 18 having a substantially elliptical or oblong fully deployed configuration can be used advantageously, as shown in FIGS. 1I-1M, which illustrate snare 10 embodiments having two loop elements 18. In other embodiments, more than two loop elements 18, such as 3, 4 or more loop elements, can be used. By matching the geometry of the deployed loop elements 18 with the geometry of the lumen, full circumferential apposition with the lumen wall can be more readily achieved. In addition, an elliptical or oblong snare 10, which can have a major axis and a minor axis, can be used in lumens having a wide range of sizes because the major axis of the snare can be rotated to provide greater wall to wall reach when needed. Additionally, the loop elements 18 can exhibit both distal and proximal reach, by forming the shape of said loops with a proximally biased curve 58, as shown in FIG. 1S. In some embodiments, the distal reach, D3, is up to about 10 mm, and the proximal reach, D4, is up to about 10 mm, where distal reach and proximal reach are in reference to the distal end of the shaft 12. In other embodiments, D3 and D4 can be greater than or less than the values recited above.

In some embodiments, each individual loop element 18 can employ a single or plurality of radiopaque markers 56, such that each loop element 18 has a different quantity of radiopaque markers 56, or a different pattern of radiopaque markers 56, to allow the operator to visually differentiate and identify each loop element 18 fluoroscopically, as shown in FIGS. 1R and 1T. For example, as illustrated in FIG. 1R, one loop element 18 has a single radiopaque marker 56 while the other loop element 18 has two radiopaque markers 56. Similarly, in FIG.

IT, the first loop element 18 has one radiopaque marker 56; the second loop element 18 has two radiopaque markers 56; the third loop element 18 has three radiopaque markers 56; and the fourth loop element 18 has four radiopaque markers 56.

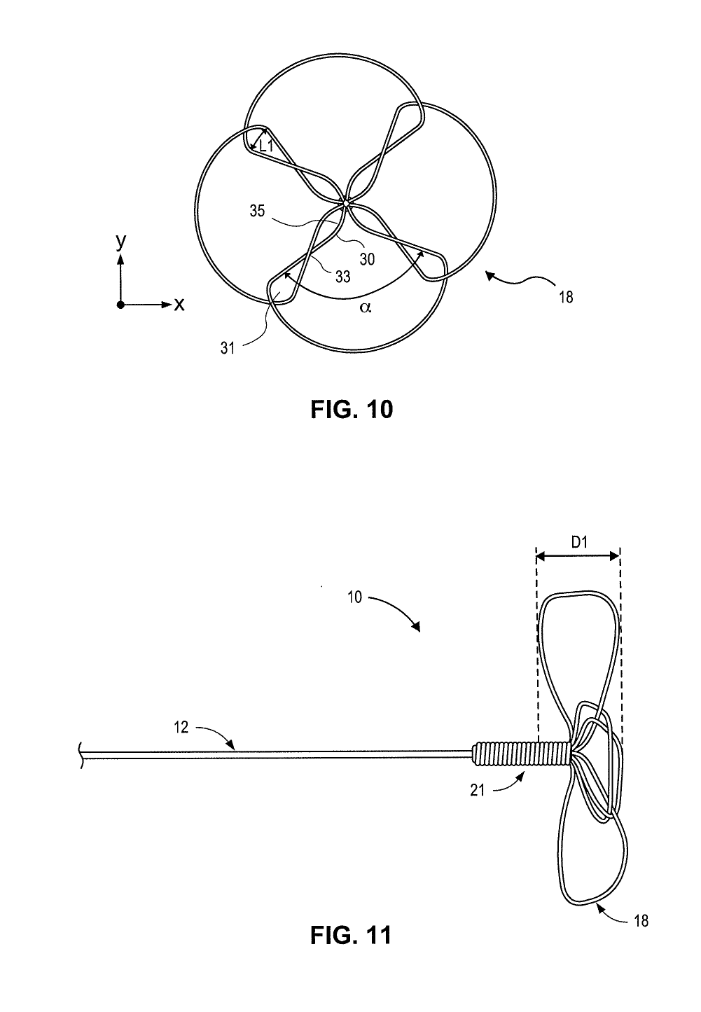

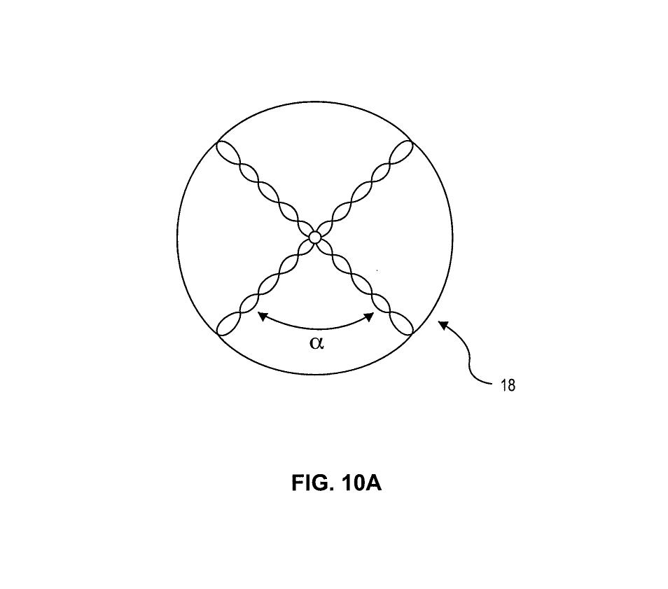

In some embodiments, the loop elements 18 can be attached or connected together using a variety of techniques, as illustrated in FIGS. 1I and 1J. For example, the loop elements 18 can be connected together by loop connectors 19 which can be made from a piece of wire, metal, plastic or polymer that can be wrapped, twisted, crimped, molded or formed around the two loop elements 18 at, for example, crossover junctions between the loop elements 18. Other techniques for connecting the loop elements 18 together can be used, such as welding or applying adhesives. Alternatively, as shown in FIGS. 1V-1X, the loop elements 18 can be connected together by loop connectors 19b which can be sleeves that are wrapped around or otherwise disposed around the adjacent spoke portions 30 of the loop elements 18. The sleeves can be made of a variety of materials, such as heat shrinkable flexible plastic tubing through which the spokes can be disposed and then secured together by shrinking the tubing around the spokes. For example, the sleeves can be made of PTFE or another biocompatible polymer. The sleeves can provide additional structural stability to the loop elements 18 and allow the loop elements 18 to be advanced or retracted in unison. Without the sleeves, the loop elements 18 may become separated, with for example one loop element facing substantially proximally and the other loop facing substantially distally, which makes control of the snare more difficult and also makes visualization of the snare and object to be retrieved more difficult. Therefore, addition of flexible sleeves, can improve control and visualization of the loop elements during the retrieval process, while still permitting the loop elements to flex and bend and be deployed and manipulated by the user. Additionally, the spoke portions 30 can be twisted together to attach the loop elements 18 together, as shown in FIG. 10A. For example, the spoke portions 30 of adjacent loop elements 18 can be twisted together. Attaching or connecting the loop elements 18 together can reduce the likelihood of unwanted or unintentional loop eversion or loop displacement that can occur during loop deployment, loop manipulation within the lumen and loop retraction.

In some embodiments, the loop elements 18 can include a single or plurality of loop collapse facilitator 23 features, as shown in FIGS. 1K-1M, that facilitates collapse of the loop elements 18 when the loop elements 18, are retracted back into the sheath 22 or when the sheath 22 is advanced over the loop elements 18. The loop collapse facilitator 23 can be a preformed crimp or fold in the loop element 18 that serves as a collapse or folding point for the loop element 18 and therefore initiates or facilitates collapse of the loop element 18 when compressive forces are applied to the loop element 18. In some embodiments, each loop element 18 can have at least one loop collapse facilitator 23.

In addition, the loop collapse facilitator 23 can be oriented in a variety ways. For example, the loop collapse facilitators 23 can be pointed or extend either in a distal direction, as shown in FIG. 1K or a proximal direction (not shown), such that the circumference of the loop elements 18 in the deployed configuration when viewed axially remains in the same shape, such as elliptical, oval or oblong, as compared to embodiments without the loop collapse facilitators 23, as shown in FIG. 1I. In other embodiments, the loop collapse facilitators 23 can be pointed or extend radially inwards as shown in FIGS. 1L and 1M, such that the circumference of the loop elements 18 in the deployed configuration when viewed axially remains in substantially the same shape, such as elliptical, oval or oblong, as compared to embodiments without the loop collapse facilitators 23, as shown in FIG. 1L. In other embodiments, the loop collapse facilitators 23 can be pointed or extend radially inwards as shown in the dotted lines in FIGS. 1L and 1M, such that the circumference of the loop elements 18 in the deployed configuration when viewed axially still remains substantially the same shape, such as elliptical, oval or oblong, but also includes a radially inward indentation, which can be arcuate and taper to a point that extends radially inwards. The size of the indentation can be controlled by the size of the loop collapse facilitator 23 as well as the shape of the taper, as illustrated by the dotted lines and solid lines representing the loop collapse facilitator in FIGS. 1L and 1M. In some embodiments, the loop collapse facilitator 23 can be oriented both distally or proximally as well as radially. In some embodiments, the loop collapse facilitator 23 can employ a loop geometry which provides a hinge point to allow the loop element 18 to fold down and collapse with low force, as shown in FIG. 1U.

FIGS. 1N-1Q illustrate the stages of deployment of an embodiment of a snare 10 with two loop elements 18. As shown in FIG. 1N, during the initial or first deployment stage, the loop elements 18 extend axially out of the sheath 22, thereby providing axial reach to the snare 10 in this configuration, which is suitable as described herein for guide wire retrieval or pacemaker lead retrieval, for example. More generally, this configuration is particularly suitable to retrieve an elongate object that is oriented transversely to the snare axis. In a second deployment stage, the loop elements 18 change from an axial orientation to a transverse or radial orientation, as shown in FIG. 10, in which the snare 10 has little or minimal axial reach. This configuration may be suitable when the space between the retrieval feature or object and another structure is small and more can more easily be accessed by loop elements with little or minimal axial reach. In the third or full deployment stage, as illustrated in FIGS. 1P and 1Q, the loop elements 18 are fully deployed, forming a circumference that is shaped to conform to the shape of the lumen, such as circular, elliptical, oval, oblong, or any other suitable shape, as illustrated in FIGS. 1I-1M. In the third deployment stage, the snare 10 can have some axial reach and full radial reach which can be configured to provide full circumferential apposition with the lumen wall. The axial reach in the third deployment stage can be increased or decreased to enhance capture of the foreign object, such as a filter, as described herein.

The diameters of the wires can be 0.002″-0.007″ each. The wires can be tightly wound together, and then formed into a loop element 18 of the desired shape. The loop element 18 outer radiused edge portion 26 can be angled such that the span of the radiused edge portion 26 is at angle of between about 45 degrees and 90 degrees, relative to the axis of the shaft 12.

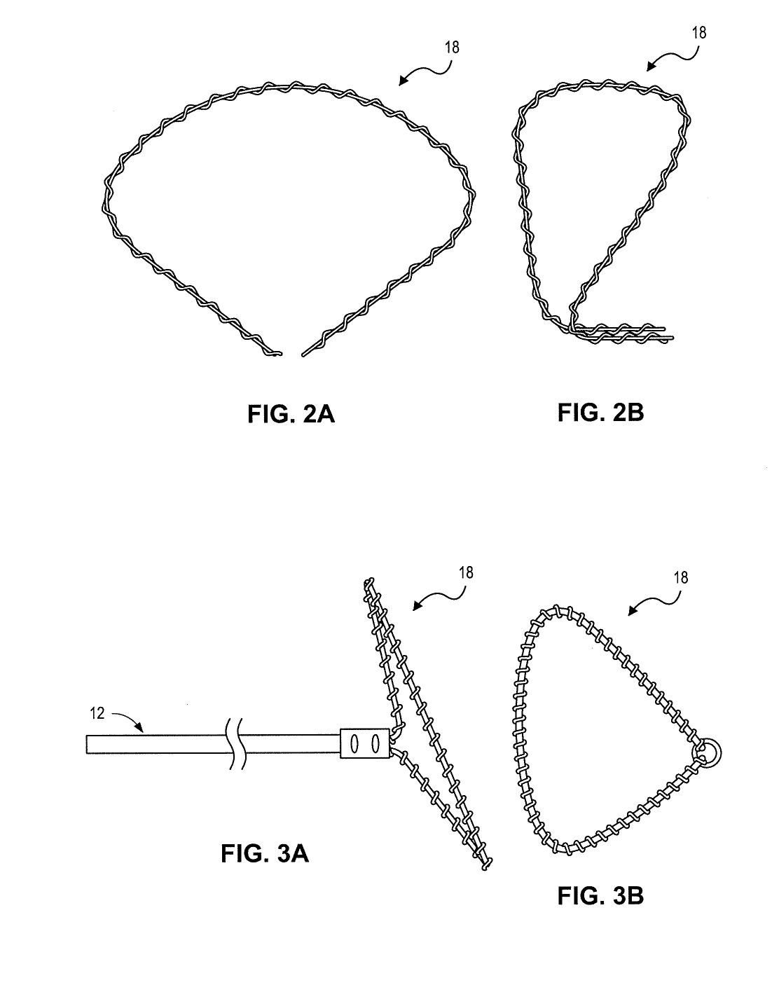

The loop element 18 of one embodiment, as illustrated in FIGS. 2A and 2B is made of at least two wires, which are tightly gathered in a twisted configuration, where at least one of the wires is a shape memory nickel titanium wire, and at least one of the wires is of a radiopaque platinum wire. In some embodiments, the twisted configuration can be advantageous over the braided configuration, when a specific stiffness property of the loop elements 18 is desired, by varying the number of wires and wire diameter used in the strand. In some embodiments, the loop element 18 includes 2 shape memory nickel titanium wires and two radiopaque platinum wires. Other materials can be used in place of the nickel titanium and/or radiopaque platinum wires. For example, the nickel titanium alloy, such as Nitinol, can be replaced with a stainless steel wire or polymeric wire. In addition, the radiopaque wire can be replaced with another radiopaque material, such as a platinum-iridium wire, a palladium wire, a gold wire, a tantalum wire, a tantalum-tungsten wire, and the like. In addition, these radiopaque materials can be incorporated into polymeric materials directly or a modified form, such as a salt for example.

The radiopaque materials can be bonded or attached to the non-opaque wire in a variety of ways, including wrapping or braiding the radiopaque wire with the non-radiopaque wire together, or by attaching marker bands to the non-radiopaque wire, or by cladding the non-radiopaque wire with the radiopaque material, for example. In many embodiments, the use of various radiopaque markers can be used to indicate the relative location and orientation of the deployed snare 10 in the target area.

FIGS. 3A and 3B depict a view of one embodiment, where just one loop element 18 is shown attached to the shaft 12 for the sake of clarity. The embodiment shown in FIGS. 3A and 3B can have a plurality of loop elements 18, such as two, three, or four loop elements 18, or more than four loop elements 18 as described herein. A snare 10 with more loop elements 18 will have more spoke portions 30 that can engage with the foreign object, which may aid in retrieval of the foreign object. However, an increased number of loop elements 18 may obscure real-time imaging of the snare elements and foreign object, making it more difficult for the operator to correctly identify all the loop elements 18 on the screen, which may interfere with efficient manipulation of the snare 10. In addition, a snare 10 with too many loop elements 18 can end up having a larger compressed diameter due to the many loop elements 18 that are attached to the shaft 12 via, for example, a hypo tube 20 swage connection, as discussed below. As more loop elements 18 are swaged to the hypo tube 20, the diameter of the hypo tube 20 increases in order to accommodate the additional loop elements 20. Increasing the compressed diameter of the snare 10 is generally undesirable for many minimally invasive techniques with which the snare 10 can be used because a larger device requires a larger percutaneous incision, which increases the pain and recovery time for the patient.

In contrast, in some embodiments a snare 10 with fewer loop elements 18, such as two loop elements 18, can be more easily visualized using real time imaging techniques, thereby allowing the operator to accurately identify each loop element 18 and therefore efficiently manipulate the position and orientation of the snare with respect to the foreign object. The two loop element embodiment, as discussed above, can still be capable of achieving complete or substantial circumferential apposition with the lumen wall. In some embodiments with too few loop elements 18, such as a single loop element, the single loop element can be too floppy, and a floppy loop element 18 can be difficult to precisely manipulate and position, making grasping a small retrieval element on a foreign object more difficult.

FIGS. 3A and 3B illustrate the shape of the loop element 18 from two angles; a transverse side view in FIG. 3A and a front axial view in FIG. 3B. The shaft 12 can be attached to the hypo tube 20 via swaging. The hypo tube 20 can also be swaged to the loop element 18. The loop element 18 can be made from a strand of four wires, two Nitinol wires and two platinum wires.

FIG. 4 is an axial view of an embodiment of a loop element 18 and a hypo tube 20. The shape of the loop element 18 includes a radiused edge portion 26 which shares its radial center with the center axis of the hypo tube 20. The radiused edge portion 26 is bounded at each end by a radiused corner feature 28, which transitions the radiused edge portion 26 into two straight spoke portions 30. These straight spoke portions 30 are typically the radius length from the central axis of the hypo tube 20 to the radiused edge portion 26 of the loop element 18. In some embodiments, the straight spoke portions 30 are set at an angle α of approximately 90 degrees, and radiate from the central axis of the hypo tube 20 to the outer radius of the radiused edge portion 26 of the loop element 18.

The loop elements 18 have a geometry that enables them to catch easily on foreign objects in the human anatomy. In some embodiments as shown in FIG. 4, the loop element 18 has a “D” shape which resembles a pie slice with rounded corners, when viewed axially along the device axis. This D shape includes a radiused edge portion 26, which shares a radial center with the axis of the shaft of the device. The radiused edge portion 26 is bounded at either end by a radiused corner portion 28 which transitions the radiused edge portion 26 into two straight spoke portions 30. In some embodiments, the radiused corner portion 28 bends about 90 degrees towards the central axis of the shaft 12.

In some embodiments, the two straight spoke portions 30, which radiate from the central axis of the hypo tube to the outer radius of the radiused edge portion 26, are set at an angle α of about 90 degrees, for a snare 10 with four loop elements 18. In some embodiments, the angle α between the two straight spoke portions 30 can be less than 90 degrees when, for example, the snare 10 has more than four loop elements 18, such as an angle of about 60 degrees for a snare 10 with six loop elements 18, or an angle of about 72 degrees for a snare 10 with 5 loop elements. To generalize, in some embodiments, the angle in degrees between the straight spoke portions 30 can be determined by dividing 360 by the number of loop elements 18 in the snare 10. This results in a configuration where the loop elements 18 cover an entire circle of space when viewed along the axial axis. Therefore, in an embodiment of the snare 10 with three loop elements 18, the angle between the two straight spoke 30 portions can be about 120 degrees. In some embodiments, the angle α between the straight spoke portions 30 can be greater than as determined using the formula set forth above, which results in an overlap of portions of the loop elements 18 with adjacent loop elements 18. In some embodiments, the angle between the two straight spoke 30 portions is greater than the value calculated in the formula set forth above, where an angle of about 5 to 15 degrees ensures that there is minimal or no gap about the perimeter of the snare, to form a closed circle.

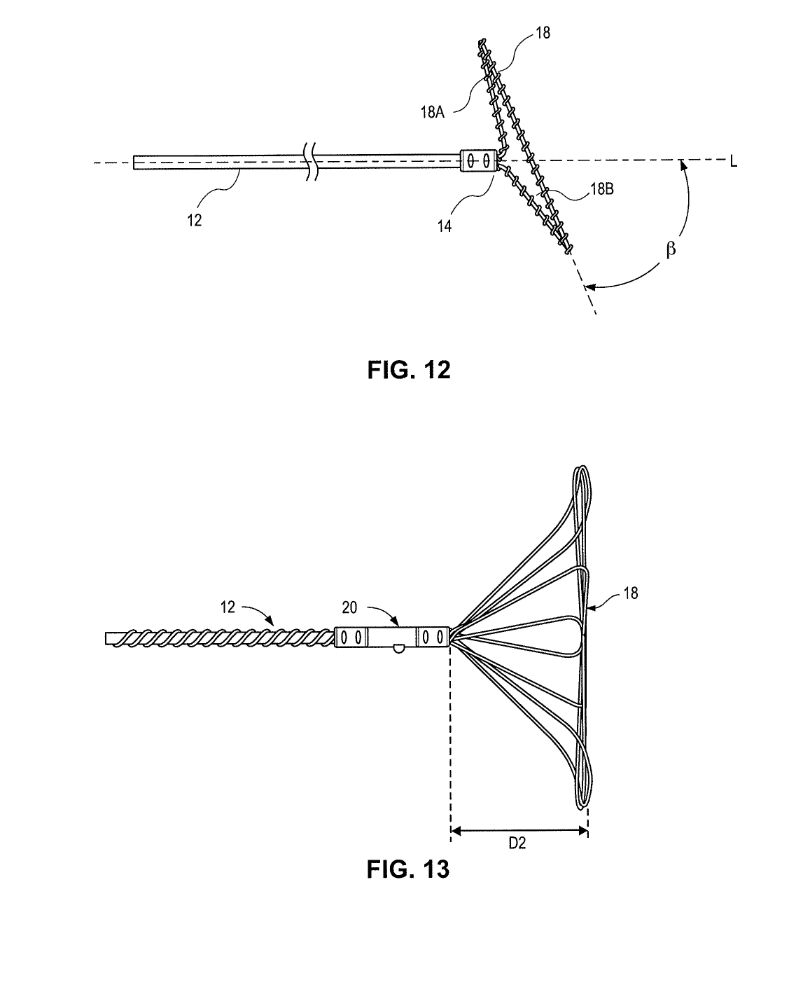

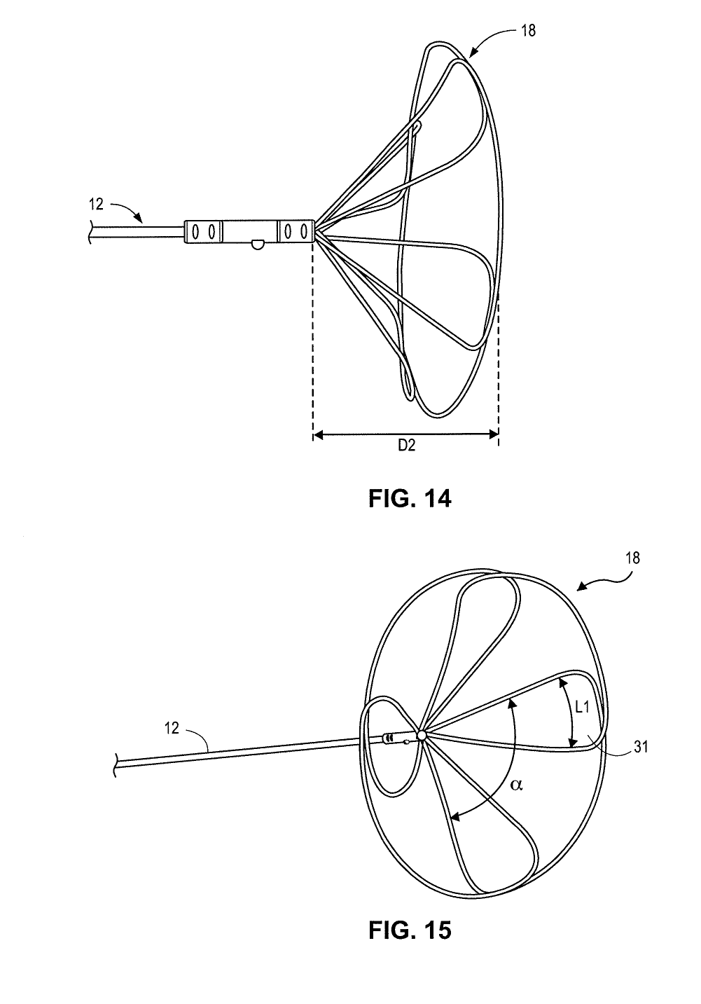

In some embodiments, from a transverse view, the large radiused edge portion 26 of the loop element 18 can be angled between about 90 degrees and about 30 degrees relative to the axis of the shaft 12 of the device 10, as shown in FIG. 12. This edge can also be substantially or exactly 90 degrees from the shaft axis, forming a flat, single plane circle when viewed transversely, as shown in FIG. 13.

In other embodiments, from a transverse view, the large radiused edge portion 26 of the loop element 18 can be angled at an angle 13 that is from about 5 to 45 degrees relative to the longitudinal axis L of the shaft 12 of the device 10, as shown in FIGS. 3A and 12. Such a configuration where the radiused edge portion 26 is angled less than 90 degrees results in a propeller like configuration where the loop element 18 has a pitch and axial reach both proximal and distal the end of the shaft 12 and/or sheath 22. As illustrated in FIG. 12, the loop element 18 has a portion proximal to the distal most portion of the shaft and a portion distal to the distal most portion of the shaft, as shown by the dotted line which divides loop element 18 into the proximal portion 18A and the distal portion 18B. In addition, the propeller configuration can result in the opening of the loop elements 18 being oriented in both a plane transverse to the snare axis and a plane parallel to the snare axis.

In these embodiments, the axial deployment length at full deployment of the loop elements 18 is relatively short when compared to some prior art devices which resemble the intermediate deployment configuration illustrated in FIG. 1F for some embodiments. A long axial deployment length can be beneficial in some situations, such as capturing a guide wire that is oriented generally transversely to the snare 10, or capturing a retrieval element on a foreign object when the retrieval element is located at or near the center of the lumen. A short axial deployment length can be beneficial in other situations, such as capturing a retrieval element that is located at or near the periphery of the lumen. In some embodiments, loop elements 18 with a long axial deployment length can inadvertently capture structural elements on the foreign object, such as frame anchors on a filter, rather than the retrieval element which is specifically designed to be engaged by the snare. When a structural element such as a frame anchor is captured instead of the retrieval element, the filter may not be able to be withdrawn into the sheath 22 and be removed. In addition, the loop elements 18 may get tangled up with the frame anchors and other structural elements more easily when the axial length is long. This can be a problem with some prior art devices, such as the EN Snare® retrieval device, which has a long axial reach. For at least these reasons, a short deployment length can be advantageous over a long deployment length in certain situations. In some embodiments, the axial deployment length of the loop elements 18 can be less than the distance between the retrieval element and the support member or anchor of the filter, thereby reducing the likelihood that the loop elements 18 will inadvertently engage the anchors on the support members. In some embodiments, the axial deployment length of the loop elements 18 can be less than the distance between the retrieval element and the support member crossover or the material capture structure of the filter. In some embodiments, the axial deployment length of the loop elements 18 can be less than the distance between the retrieval element and any structure on the filter in which the loop elements can get entangled with or that interfere with the function of the loop elements 18.

In addition to the axial deployment length, loop elements of prior art devices lack substantially complete circumferential apposition with the vessel wall, which makes it difficult to retrieve objects near the periphery of the blood vessel lumen. In contrast, embodiments of the snare disclosed herein achieve substantially complete circumferential apposition which facilitates retrieval of objections, such as retrieval elements on filters, that are located near the periphery of the blood vessel lumen.

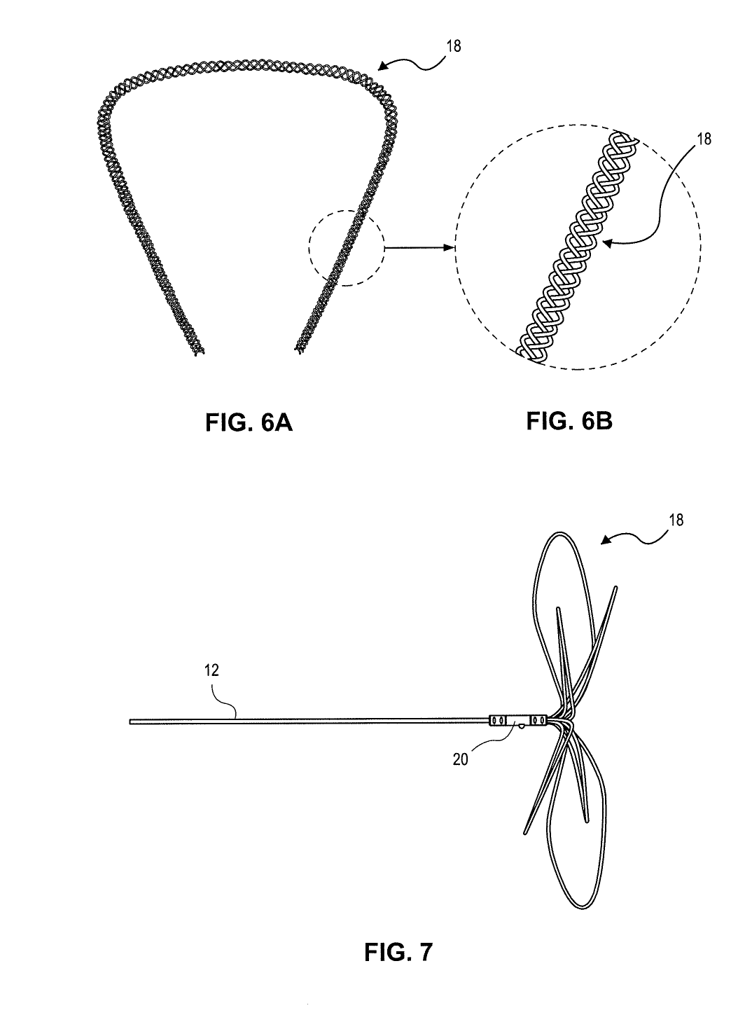

FIGS. 5A and 5B illustrates an embodiment of a loop element 18 made of four round wires, which are tightly gathered in a twisted configuration, where two of the wires are of shape memory nickel titanium wire, and two of the wires are of a radiopaque platinum wire. The diameters of the wires can be about 0.004″ each. The wires are tightly wound together, and then formed into a loop shape. In some embodiments, the loop outer radius is angled such that the span of the radius is at angle of between about 45 degrees and 90 degrees, relative to the axis of the shaft. FIGS. 6A and 6B illustrates a similar embodiment of a loop element 18 made of four wires, except that the wires are braided together rather than twisted together to form the loop element 18.

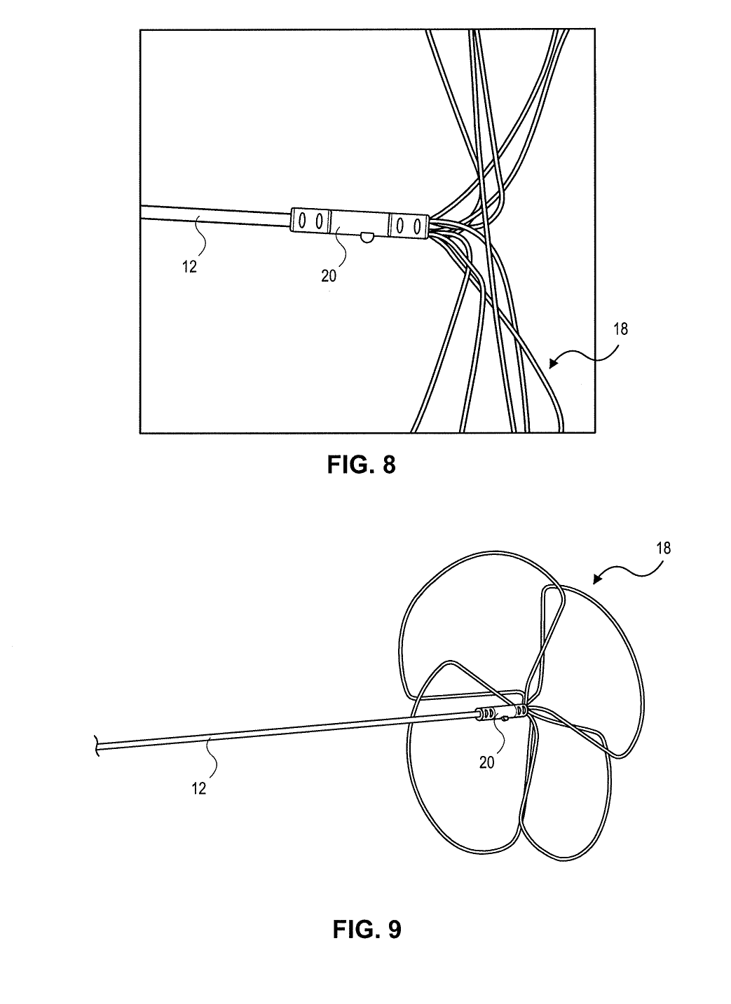

One alternate embodiment of the device 10, illustrated in FIGS. 7-10, includes a series of loop element structures 18 mounted in a substantially circular geometry when viewed along the longitudinal axis. In some embodiments, the loop elements 18 extend substantially transversely with respect to the longitudinal axis. In some embodiments, the outer circular perimeter defined by the loop elements 18 is substantially continuous and does not have gaps. In some embodiments, the overlap 31 between the loop elements 18 is as described above for FIG. 1A, where the overlap 31 covers a pie shaped region that extends from the outer circumference of the loop elements to the center where the loop elements are attached to the shaft. In other embodiments, the overlap 31 between the loop elements 18 can change as the loop elements 18 are further extended out of the sheath. For example, as shown in FIG. 10, the loop elements 18 can have an overlap 31 that occurs over approximately the middle to distal portion of the loop elements 18. As illustrated in FIG. 10, the overlap 31 begins at crossover points 33 between the spokes 30 of the loop elements 18. In some embodiments, as the loop elements 18 are retracted back into the sheath, the crossover points 33 move closer towards the center, until the crossover points merge into the center, resulting in an overlap configuration similar to that illustrated in FIG. 1A. In addition to the variable overlap regions, the embodiment illustrated in FIG. 10 has interior gap portions 35 between the loop elements. These interior gap portions 35 extend radially inwards from the crossover points 33, and can decrease in size and disappear as the loop elements 18 are retracted back into the sheath. In these embodiments, the loop elements 18 can have a radial span that can be defined by the angle α, and an overlap with a span L1, similar to that described above for FIG. 1A. In these embodiments and in others, the overlap portions can also act as additional snaring portions which increase the likelihood that a portion of the device engages the object to be retrieved.

In some embodiments, the loop elements 18 can be attached to a shaft 12 via a swaged or crimped hypo tube 20. These loop elements 18 can be made of two or more wires, including at least one Nitinol wire and at least one platinum wire. As illustrated in FIGS. 7-10, in some embodiments the most distal part of the device 10 can be the loop elements 18 because the device 10 does not have a distally extending control member that can be found in some prior art devices, such as the grasping device disclosed in U.S. Pat. No. 7,753,918. In some embodiments, the presence of a control member may interfere with retrieval of the foreign object, such as a filter, by getting entangled with the filter, making it advantageous for some embodiments to not have a distally extending control member. In some embodiments, the loop elements 18 can be angled or have a pitch with respect to the longitudinal axis.

FIG. 11 illustrates another embodiment of the snare 10 where the loop elements 18 are attached to the shaft 12 with a wire coil 21. In some embodiments, the wire coil 21 can be a separate wire that can be wrapped around the proximal portions of the loop elements 18. In other embodiments, the proximal portions of the loop elements 18 can be wrapped around the distal end of the shaft 12 in order to form the wire coil 21. As additionally shown in FIG. 11, the loop elements 18 can extend axially, or in other words, have an axial depth, D1, that can be between about 1 to 10 mm. This axial reach allows loop elements 18 to effect capture of an object, such as a retrieval element of a filter, via rotation about the longitudinal axis of the snare. In some embodiments, the axial depth, D1, is less than the distance between a retrieval element on a filter and the closest anchor to the retrieval element, as further described below.

Another alternate embodiment, as illustrated in FIGS. 13-15, utilizes a twisted strand shaft 12 made of four 0.010″ Nitinol wires. This shaft 12 is attached to twisted strand loops elements 18 using a hypo tube 20 using silver solder, for example. After full deployment, the loop elements 18 form a substantially circular geometry which is in a single plane typically 90 degrees from the axis of the shaft 12. In some embodiments, as illustrated, the loop elements 18 extend both transversely and axially with respect to the longitudinal axis of the shaft 12, forming a cone-like structure with a circular base defined by the distal edge portions of the loop elements 18. The axial reach, D2, or extension of the circular portion past the distal end of the shaft can vary and can depend on and be less than, for example, the distance between the retrieval element and a particular filter structure, such as an anchor, support member, support member crossover, or material capture structure of the filter, as further described herein. The axial reach, D2, can be between about 1 to 10 mm. In addition, the loop elements 18 can a region of overlap 31 and can have a radial or circumferential span defined by the angle α, as described above with reference to FIGS. 1A and 4.

In some embodiments, this design offers several key features and capabilities, for example:

1. Loop Design

The design of the loop elements allows for deployment in different size lumens, and can conform to variations in lumen anatomy such as tapering, curvature, and angulations. This conformance feature can also enable the device to achieve full radial apposition with the target lumen regardless of lumen diameter or circularity. The loop configuration allows the device to catch a foreign object no matter where the object is located within the luminal space, since the loops reach full radial apposition within the lumen. The design of the elements allows the snare to fit into a very small guiding sheath, facilitating navigation through tortuous anatomies. The angled design of the loop radius allows the device to have axial reach both distal and proximal to the point where the loops are attached to the shaft, enabling the loops to locate foreign objects with minimal forward and backward axial manipulation of the device by the user. The non-angled design of the loop radius allows the device to have a flat, single plane circle geometry, enabling the loops to locate foreign objects with which may be against the vessel wall or partially embedded in the vessel wall. The loops can be made radiopaque, which allows visualization of the loop under fluoroscopy. Additionally, each individual loop element can employ a single or plurality of radiopaque markers such that each loop element has a different quantity of radiopaque markers, or a different pattern of radiopaque markers, to allow the operator to visually differentiate and identify each loop element fluoroscopically.

2. Shaft Design

The diameter and mechanical properties of the shaft, such as tensile strength, stiffness and/or elasticity, allows the user to manipulate the loops easily, by transferring axial and torsional motion from the proximal end of the device down to the distal end of the device. The diameter of the shaft allows for it to fit within a small diameter guiding sheath. The diameter of the shaft provides tensile support and strength to allow for high forces that may be required for removing a foreign object from the human anatomy. The shaft can be either solid or hollow, allowing the passage of devices, such as a guidewire, through the shaft. The shaft can be of a single element such as a wire, or a construction of a plurality of elements which are braided or stranded together. The shaft can be of a radiopaque material, to facilitate fluoroscopic visualization.

3. Hypo Tube Design

The inner diameter of the hypo tube allows the loop wires and shaft wire to fit snugly within the inner diameter, to facilitate mechanical swaging, soldering, or crimping of said hypo tube, mechanically locking the elements together. The outer diameter of the hypo tube provides adequate wall thickness to allow mechanical swaging or crimping of the hypo tube to provide a strong mechanical attachment, without cracking the hypo tube. The hypo tube can be of a radiopaque material, to facilitate fluoroscopic visualization. Additionally, the hypo tube can be radially shaped into a non-circular shape, such as but not limited to a hexagon or square or rectilinear shape, to further facilitate mechanical fit and locking of the shaft and loop elements.

In some embodiments, the fundamental design elements which achieve these features include, for example: (1) a plurality of loop elements, which are attached to a shaft via a hypo tube; (2) loops which are designed to be flexible and radiopaque; (3) loops which can be collapsed within a guiding catheter, and deployed outside of the guiding catheter; (4) loops which can reach full circular apposition within the luminal space in a human body; (5) loops which are attached to a shaft distally, which extend laterally towards the wall of the vessel of a human body; (6) loops which are angled relative to the axis of the shaft, typically less than 91 degrees and typically greater than 1 degrees; (7) loops which employ an attachment that is typically a crimped or swaged hypo tube; (8) a shaft which is attached to the loops; (9) a shaft having a diameter allows it to fit within a small diameter guiding catheter, (10) a shaft which can be either solid or hollow; (11) a shaft made of a material which can be polymeric, or can be of a metal such as but not limited to nickel titanium; and (12) a shaft having a length designed to enable the user to position the loops at a desired location to remove a foreign object from a human body.

In some embodiments, the snare device 10 is designed for placement into a guiding sheath 22, being advanced through said sheath 22, deploying near a foreign object located within the human anatomy, capturing said object, and removing the object from the human anatomy. The shape of the loop elements 18 allows them to conform to the diameter of the vessel in which they are deployed into, allowing easier capture of the foreign body with less manipulation.

The device 10 enables a user to capture a foreign object located within the human anatomy, grasp said object in a controlled manner, and retrieve and remove said object from the human anatomy. Examples of foreign objects which might be removed from the human anatomy include implants such as stents, guidewires, leads, filters, and valves, and organic objects such as kidney stones or calcified emboli. For example, a snare 10 can be used to capture a vena cava filter and pull it into a retrieval sheath 22 for removal from the patient.

FIGS. 16-19 illustrate embodiments of methods of using any of the snares 10 disclosed herein. As shown in FIG. 16, the snare 10 can be advanced through one or more retrieval sheaths 22 and up to the site of a deployed filter 40, which, for example, can be located within the lumen 46 of a blood vessel 48. In some embodiments, the snare 10 can be pre-loaded into a sheath 22 which can be inserted into the patient via a minimally invasive procedure, such as a percutaneous insertion technique. In some embodiments, the distal end 24 of the sheath 22 can be advanced to or proximally to the retrieval element 42 of the filter 40. In some embodiments, the distal end 24 of the sheath 22 is advanced just past, i.e. just distal, the retrieval element 42, taking care to avoid advancing the distal end 24 into the other elements of the filter 40, such as the filter portion 44 or anchors 50 on the filter frame 52, which would indicate that the distal end 24 had been advanced too far. In some embodiments, the distal end 24 is advanced to a location distal the retrieval element 42 and proximal the anchors 50 closest the retrieval element 42. In some embodiments, the sheath 22 includes a radiopaque marker 54 located near the distal end 24 of the sheath 22 that facilitates alignment of the distal end 24 with respect to the filter 40. For example, the operator can align the radiopaque marker on the sheath 22 with the radiopaque retrieval element 42 of the filter 40 under fluoroscopy, which results in the distal end 24 of the sheath being correctly positioned for loop element 18 deployment, which in some embodiments as described herein is located between the retrieval element 42 and the anchor 50 closest to the retrieval element.

As illustrated in FIG. 17, the snare 10 is then deployed into the vessel 48. As described above, deployment of the snare 10 can include three deployment phases. In some embodiments, deployment of the snare 10 can include less than three deployment phases, such as one or two deployment phases, while in other embodiments, deployment of the snare 10 can include more than three deployment phases. FIG. 17 illustrates full deployment of the snare 10 into the vessel 48 with the loop elements 18 in a propeller-like configuration that provides some axial reach both proximal and distal to the distal end 24 of the sheath 22. In some embodiments, the axial reach in the distal direction can be less than the distance d between the retrieval element 42 and anchor 50, thereby reducing the likelihood that the loop elements 18 become entangled with or caught on the anchor elements 50 of the filter during loop element 18 deployment and manipulation. In some embodiments, the distance d can be between about 5 to 20 mm The region between the retrieval element 42 and the anchor 50 forms a zone of action in which the loop elements 18 can be deployed and manipulated to effect capture of the retrieval element 42. In some embodiments, the loop elements 18 can have a pitch like the blades of a propeller such that the openings of the loop elements 18 are oriented in both a plane transverse to the snare 10 axis and a plane parallel to the snare axis. This allows the loop elements 18 to capture the retrieval element 42 either by moving the loop elements 18 axially in a proximal or distal direction or by rotating the loop elements 18 about the snare axis. In some embodiments, the loop elements 18 are deployed distal the retrieval element 42 and proximal the support member of the filter, such that the loop elements 18 achieve substantial apposition with the full circumference of the lumen wall, which is advantageous for capturing retrieval elements located near the periphery of the lumen. The deployed loop elements 18 can be withdrawn or retracted proximally to engage the retrieval element.

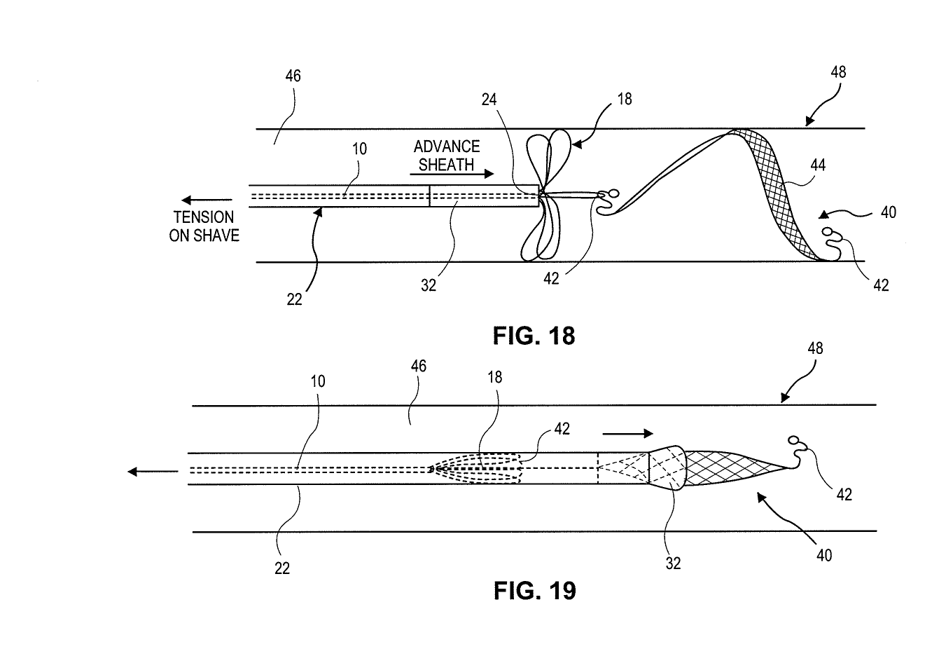

FIGS. 18-19 illustrate the loop element 18 engaged with the retrieval element 42 of the filter 40 and the subsequent collapse of the filter 40 into the sheath 22. After the retrieval element 42 is secured, the snare 10 is held under tension while the sheath 22 is advanced over the filter 40, thereby collapsing the filter 40 into the ID of the sheath 22. In some embodiments using both an inner sheath 22 and an outer sheath, the retrieval element 42, and optionally a portion of the filter 40, is first retracted or pulled into an inner sheath 22, in order to secure the filter 40 to the snare 10 and to prevent or reduce unfurling of the tail portion of the filter 40, before the outer sheath is advanced over the rest of the filter 40.

As the sheath 22 is advanced over the filter 40, the flexible distal tip portion 32 of the sheath 22 can expand and invert over the filter 40, providing a ramp in which the filter 40 can be drawn into the sheath 22. In some embodiments, the inversion of the distal tip portion 32 can be initiated by contact with specific structures on the filter, such as the retrieval element and/or anchors on the filter frame. In some embodiments, the snare 10 can be retracted in the proximal direction while the sheath 22 is advanced in the distal direction to capture the filter 40 within the sheath 22. In other embodiments, the snare 10 can be retracted in the proximal direction while the sheath 22 is held relatively immobile, i.e. neither advanced nor retracted, to capture the filter 40 within the sheath 22. In some embodiments, the entire filter 40 can be retracted into or captured by the inner sheath.

Another example is the use of a snare 10 to grasp and extract loose kidney stones from a patient's kidneys. The snare 10 is advanced through one or more sheaths 22, up to the site of the loose kidney stone. The snare 10 is then deployed and engaged with the stone. Next, the snare 10 is pulled into the sheath 22, or the sheath 22 advanced over the snare 10, drawing the stone into the distal ID of said sheath 22.

As described above, the retrieval system can include a plurality of different components, such as a guide wire, a snare 10, an inner sheath and an outer sheath 22. The proximal ends of these components are generally located outside the patient's body so that the operator can manipulate each of the components by grasping the proximal portion of the components and moving the component in a proximal or distal direction. Often, the proximal portions or ends of the components are or can be reversibly secured or fixed to one another in a proximal handle portion, using a rotatable or twist fitting, such as a luer lock, for example. Because one hand of the operator is often used to manipulate the component, only one hand is free to disconnect or connect the fittings, which can be difficult to do for a rotatable luer lock fitting. In addition, the twisting or rotation of the twist fitting can lead to unintentional and undesired twisting or rotation of the snare device.

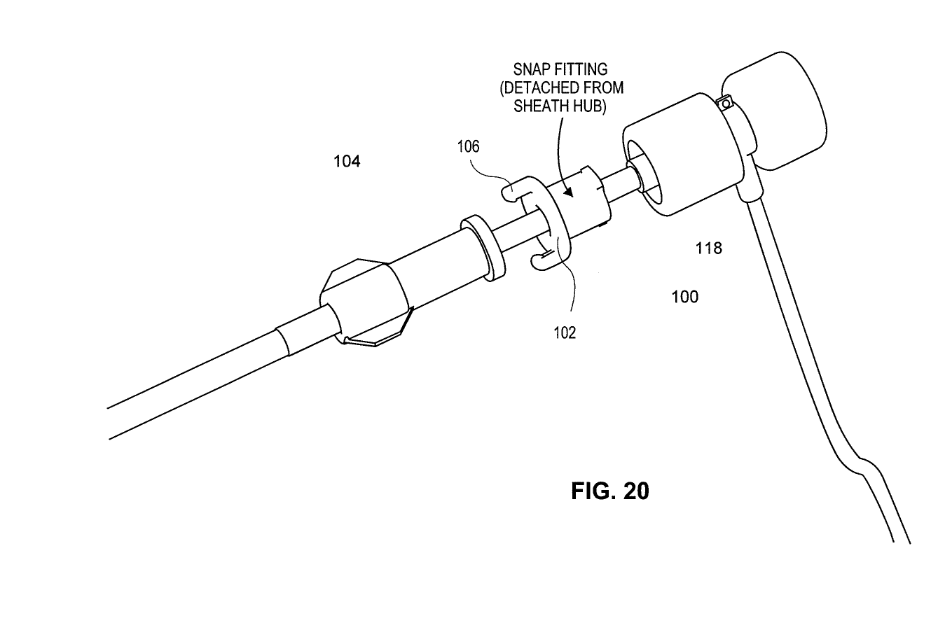

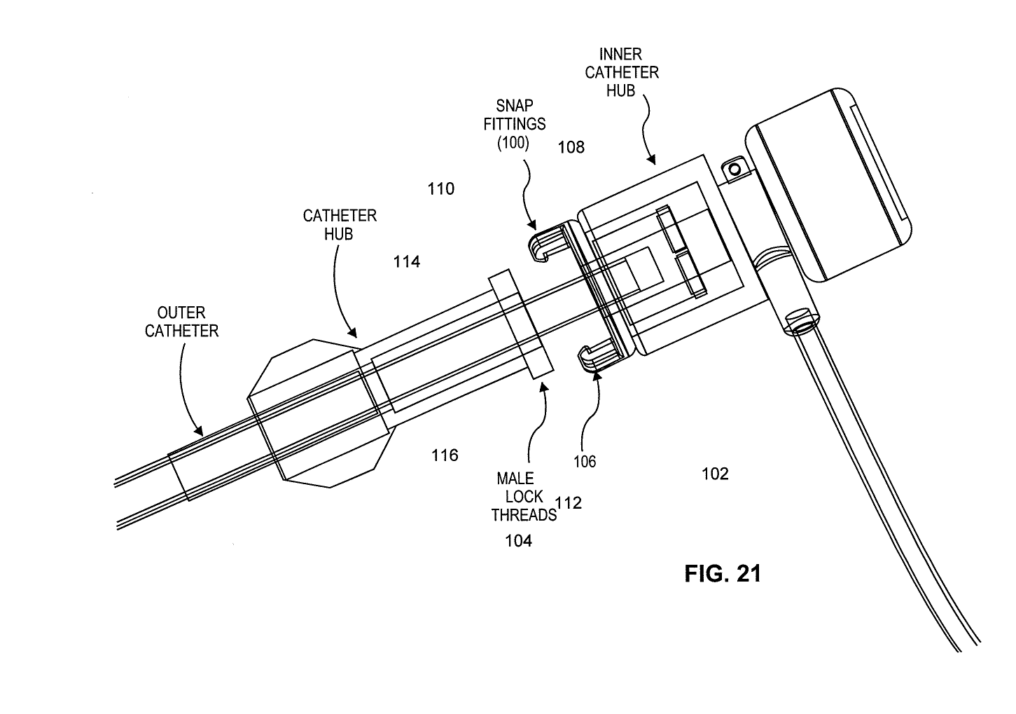

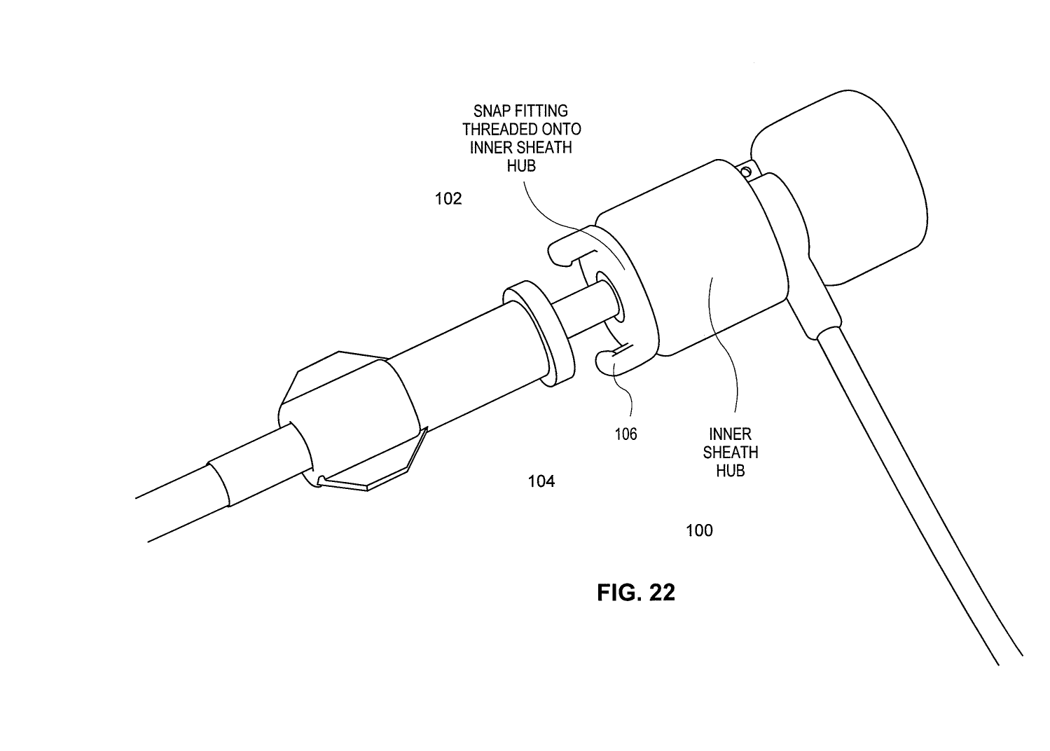

Therefore, it would be advantageous to provide fittings that can more easily be manipulated with one hand, such as a snap fitting, as illustrated in FIGS. 20-22. The snap fitting 100 comprises a female connector 102 and a male connector 104. In some embodiments, the female connector 102 can have a plurality of flexible latch portions 106 that define an opening 112 and enclose a receptacle 108 that is configured to receive the male connector 104. For example, the female connector 102 can have 2, 3, 4 or more latch portions 106. The distal end of each flexible latch portion 106 can include a retaining feature 110 that projects radially inwards and functions to secure the male connector 104 within the receptacle 108. The male connector 104 comprises a distal portion 114 that is configured to fit through the opening 112 and within the receptacle 108. The male connector 104 can also include a narrow stem portion 116 that has a diameter less than the diameter of the opening 112. In some embodiments, the distal portion 114 and/or the latch portions 106 can be tapered towards the outer or inner edge in order to present an angled surface to the opening 112 that can aid in widening the opening 112 by pushing apart the latch portions 106.

These snap fittings 100 can be integrated into the proximal ends of the various components described herein, and well as other components that can be used with the retrieval system. Alternatively, the snap fittings 100 can be made into luer lock adaptors, or other connector adaptors such as screw adaptors, that allow the operator to convert a luer lock fitting, or other fitting, into a snap fitting, as illustrated in FIGS. 20-22. In some embodiments, the device can include an outer catheter with an outer catheter hub and an inner catheter with an inner catheter hub. The female connector 102 of the snap fitting 100 can include a locking feature 118, such as a luer lock fitting, that allows it to reversibly attach to the inner catheter hub. The outer catheter hub can include the male connector 104, which can be integrated into the outer catheter hub as illustrated, or can be reversibly attached as described above for the female connector 102. In some embodiments, all the components are locked together during insertion.

In some embodiments, the proximal gripping portions of the components can include an indicator that identifies which component the operator is gripping, thereby reducing the confusion that can occur in locating the corresponding proximal gripping portion for the desired component. In some embodiments, the gripping portion can include a visual indicator. For example, the different components can have color coded gripping portions, or can be labeled with, for example, an easily read symbol or the name of the component. In some embodiments, the gripping portion can include a tactile indicator that allows the operator to distinguish between the different components without having to look at the gripping portions, which allows the operator to maintain visual focus on more important matters, such as real-time imaging of the retrieval system within the patient provided through fluoroscopy. For example, one component can have a smooth gripping portion, another component can have a rough or knurled gripping portion, and another component can have a dimpled or ridged gripping portion. Each component can have a different tactile pattern to provide tactile contrast between the components.

In some embodiments, a pressure sensor and/or an intravascular ultrasound (IVUS) transducer can be added to or incorporated into the delivery system and method. The pressure sensor can be used to measure the pressure at various positions within the vasculature, which can be used to determine blood flow, while the intravascular ultrasound (IVUS) transducer can be used to measure fluid flow and/or provide imaging within the vessel. In some embodiments, the pressure sensor and/or IVUS transducer can be incorporated into the guidewire at one or more locations, such as the distal end or distal portion of a guidewire, as described in U.S. Pat. No. 8,277,386, U.S. Pat. No. 6,106,476 and U.S. Pat. No. 6,780,157 which are hereby incorporated by reference in their entireties for all purposes, as well as being incorporated into intermediate and proximal portions of the guidewire. The guidewire with the pressure sensor and/or the IVUS transducer can be used much like a normal guidewire to help navigate the delivery device through the vasculature, with the added benefit of providing pressure measurements and ultrasound imaging to help in the navigation, to visualize the device placement site, and to monitor and ensure proper device deployment. In some embodiments, the IVUS transducer generates image slices as it is advanced and retracted which can then be assembled together to form a three dimensional reconstruction of the vasculature and/or the device within the vasculature. In some embodiments, the guidewire with the pressure sensor and/or IVUS transducer can be fastened to a catheter in a similar manner to that described below for a catheter having a pressure sensor and/or IVUS transducer that is fastened to another catheter.

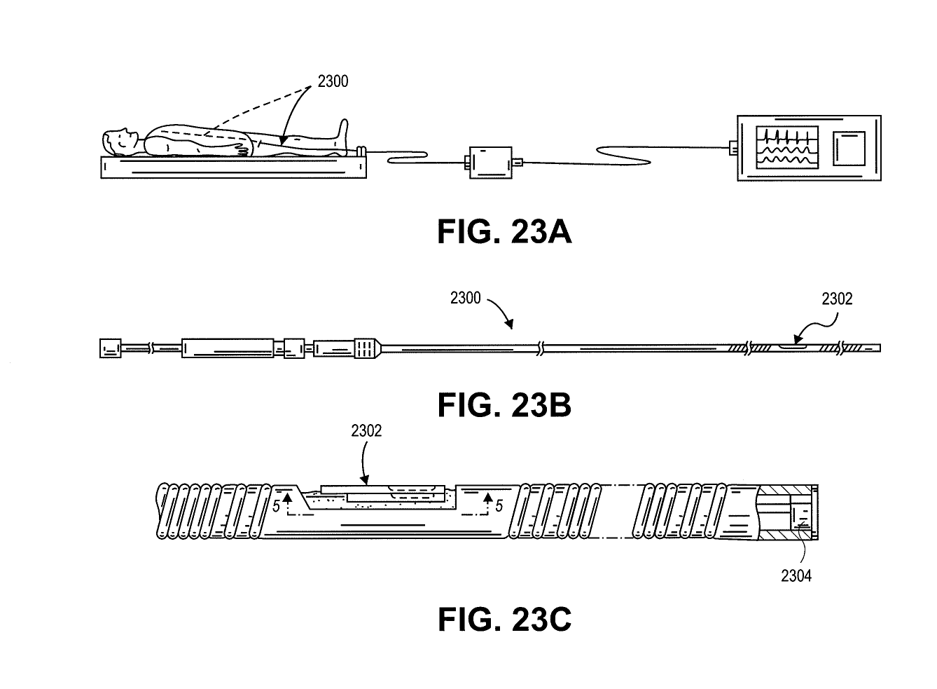

FIGS. 23A-23C illustrate an example of a guidewire 2300 having both a pressure sensor 2302 and an IVUS transducer 2304 located at the distal portion of the guidewire 2300. In some embodiments, the pressure sensor 2302 can be made from a semiconductor material, such as silicon, that is formed into a diaphragm and can be located proximally of the distal tip, while the IVUS transducer 2304 can be located at the distal tip of the guidewire 2304.

In some embodiments, the pressure sensor and/or IVUS transducer can be located on a catheter in a similar configuration to the guidewire. For example, the IVUS transducer can be located on the distal tip of the catheter while the pressure sensor(s) can be located proximally of the IVUS transducer at one or more locations along the catheter body, from the distal portion of the catheter to an intermediate portion of the catheter to the proximal portion of the catheter. The pressure and/or imaging catheter can be used in parallel with the delivery or retrieval device or any other catheter that is inserted into the vasculature. In some embodiments, the pressure and/or imaging catheter can be fastened to the delivery or retrieval device or other catheter by, for example, enclosing both catheters in a sheath or larger catheter or by fusing the two catheters together. For example, U.S. Pat. No. 6,645,152 and U.S. Pat. No. 6,440,077, both to Jung et al. and hereby incorporated by reference in their entireties for all purposes, discloses an intravascular ultrasound catheter joined together in parallel with a vena cava filter delivery device to guide placement of the filter in the vena cava. The pressure and/or imaging catheter can be used for the same purposes as the pressure and/or imaging guidewire.

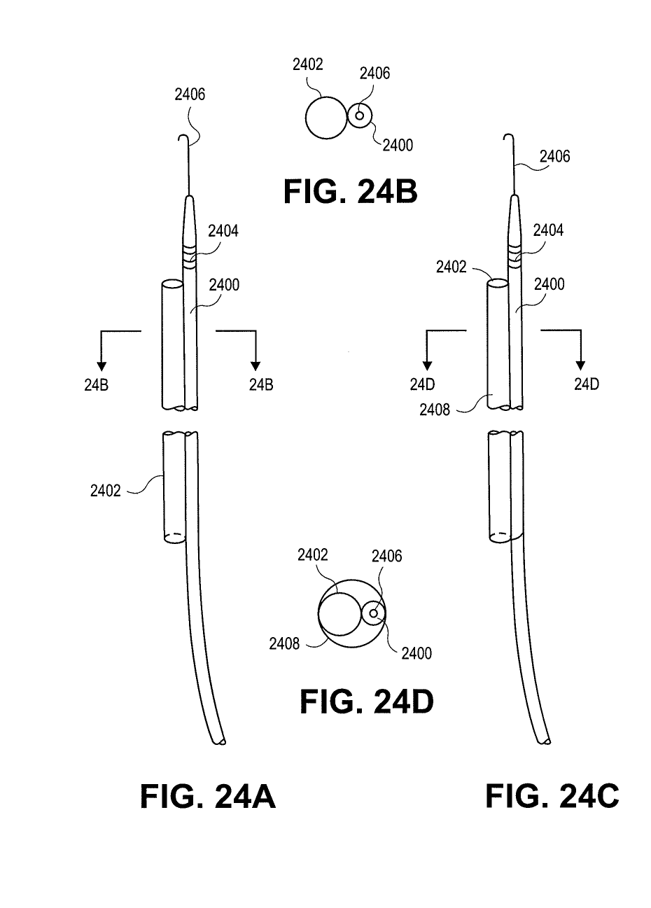

FIGS. 24A-24D illustrate two embodiments of an intravascular ultrasound catheter 2400 joined together in parallel with a catheter 2402 that can be used, for example, to deliver a device to a location with the vasculature, such as a vena cava filter to the vena cava. The intravascular ultrasound catheter 2400 can have an IVUS transducer 2404 located on the distal portion of the IVUS catheter 2400. The IVUS transducer 2404 can be a solid state transducer that is disk shaped or cylindrically shaped with a hole to allow passage of a guidewire 2406 or other device through the IVUS catheter 2400. As shown in FIGS. 24A and 24B, the IVUS catheter 2400 and the delivery catheter 2402 can be joined together in parallel without a sheath by adhering or fusing the two catheters together. FIGS. 24C and 24D illustrate the same IVUS catheter 2400 and delivery catheter 2402 fastened together using a sheath 2408.

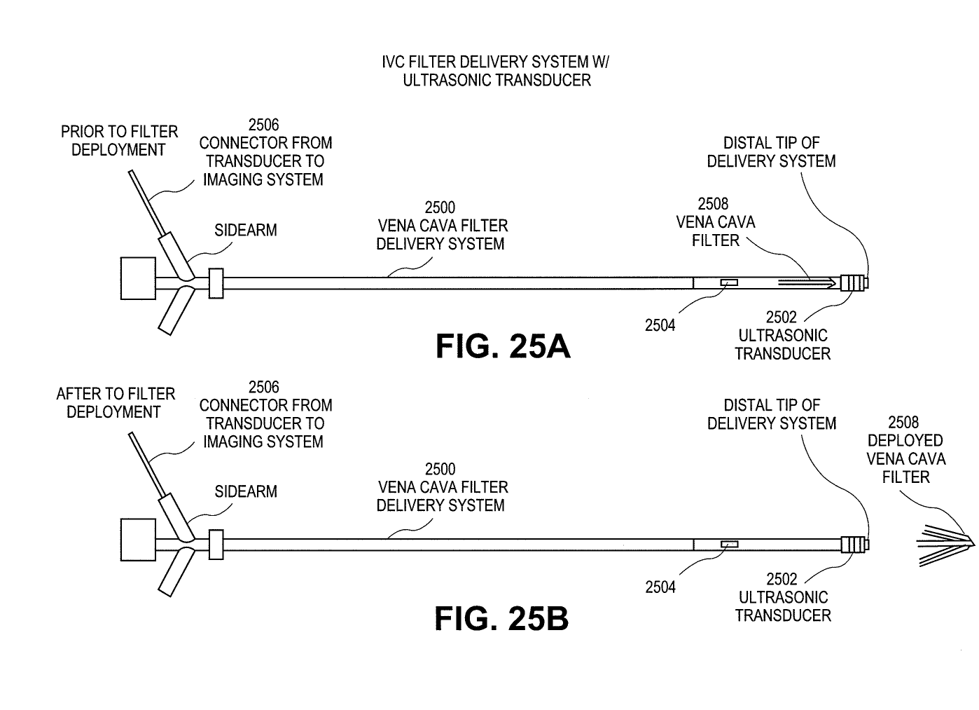

In some embodiments as illustrated in FIGS. 25A and 25B, the pressure sensor and/or IVUS transducer can be integrated into the delivery or retrieval catheter 2500 or device itself. For example, the IVUS transducer 2502 can be integrated into the distal tip or end of the catheter 2500 or device. The pressure sensor 2504 can be located on a distal portion of the catheter shaft proximally of the IVUS transducer 2502. A wire can extend from the IV US transducer 2502 and/or pressure sensor 2504 to one or more connectors 2506 located at the proximal end of the catheter 2500. The connector(s) 2506 can be used to connect the IVUS transducer 2502 and/or pressure sensor 2504 to an imaging system and/or processing system. In the illustrated embodiment, the catheter 2500 can be used to deliver a vena cava filter 2508 to the vena cava. The catheter 2500 can additionally have a telescoping sleeve or pusher rod to deploy the vena cava filter 2508, or alternatively, the outer catheter sheath can be retracted to deployed the filter. The IV US transducer can provide positioning guidance and determine the relative location of the filter by advancing and retracting the IVUS transducer 2502 on the catheter 2500 to generate a plurality of image slices that can be assembled to reconstruct a three dimensional image.

Use of the ultrasound imaging system allows the operator to deliver the device without fluoroscopy or using less fluoroscopy, thereby reducing the radiation exposure to the patient, while allowing more accurate evaluation of the vasculature, aiding placement of the device and allowing confirmation that device placement was proper. The imaging can be used to aid in the deployment of the filters or other devices. The imaging can also be used to aid in the retrieval of the deployed devices by providing visualization of, for example, the retrieval features on the deployed device and of the retrieval features, such as loops on a snare, of the retrieval device. The vasculature and implant location can be imaged prior to deployment, after deployment and/or during deployment. The imaging can be used during the retrieval process. The imaging can be used to aid in positioning of the filter or device within the vasculature. The imaging can be used to image the deployment location and determine the appropriate sizing of the filter or other device. The imaging can be used to help estimate treatment duration.

Although an imaging systems described above have been ultrasound based, other imaging systems can be used instead or in addition. For example, the imaging system can be based on intravascular ultrasound (IVUS), Forward-Looking IVUS (FLIVUS), optical coherence tomography (OCT), piezoelectric micro-machined ultrasound traducer (PMUT), and FACT.

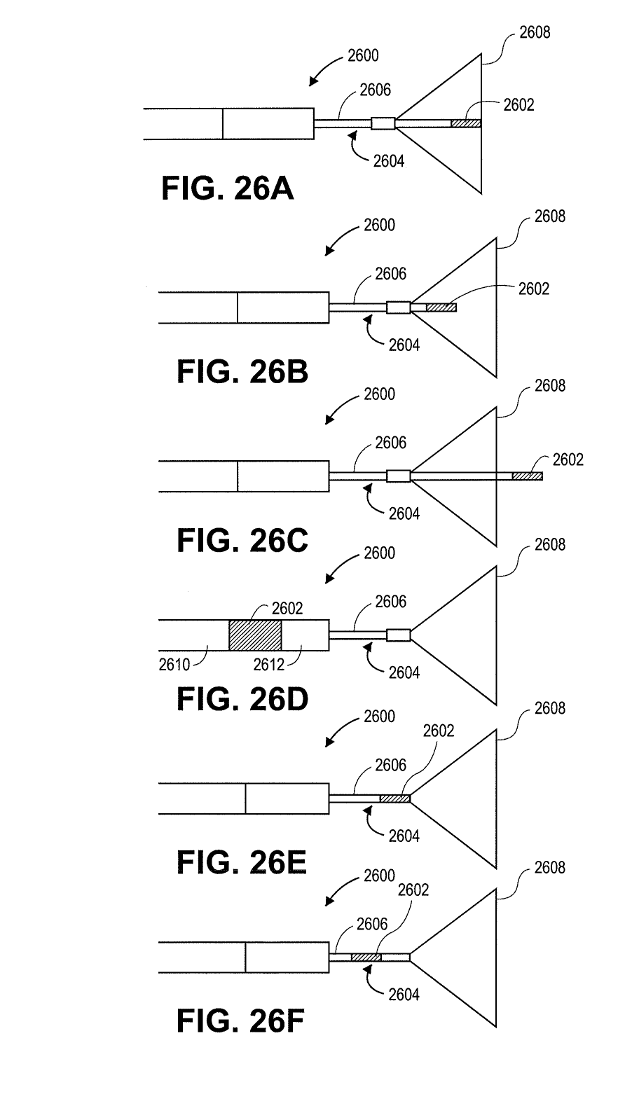

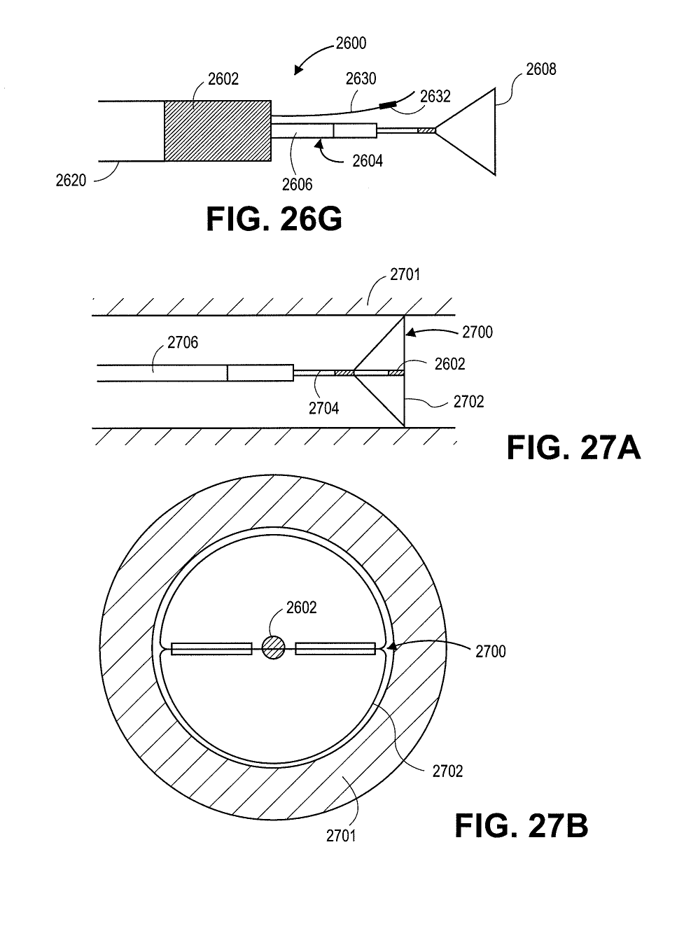

FIGS. 26A-26G illustrate various embodiments of a retrieval device and/or system 2600 that can include an IVUS transducer 2602 for imaging a deployed device, such as a filter, within the lumen of a vessel. In some embodiments, the retrieval system 2600 can have a plurality of IVUS transducers 2602 located in any of the positions as described herein. In some embodiments, as described above, the retrieval system 2600 includes a snare 2604 having shaft 2606 and a plurality of loop elements 2608 attached to the distal portion of the shaft 2606. In some embodiments, the loop elements 2608 extend both axially and radially outwards.

In some embodiments, as illustrated in FIGS. 26A-26C, the loop elements 2608 can be attached to the shaft 2606 proximally of the distal end of the shaft. An IVUS transducer 2602 can be located on the distal end of the shaft 2606. As shown in FIG. 26A, the loop elements 2608 can be attached to the shaft 2606 such that the distal ends of the loop elements 2608 when fully deployed are aligned or substantially aligned with the IVUS transducer 2602. In other embodiments, as illustrated in FIG. 26B, the distal ends of the loop elements 2608 when fully deployed are located distally of the IVUS transducer 2602. In other embodiments, as illustrated in FIG. 26C, the distal ends of the loop elements 2608 when fully deployed are located proximally of the IVUS transducer 2602. These configurations can be used to optimize both the ability of the IVUS transducer to image the retrieval feature of the filter and the ability to align the distal end of the loop elements 2608 with the retrieval feature of the filter. A variety of factors can dictate which configuration is appropriate, such as the configuration of the retrieval feature and the imaging capability and configuration of the IVUS transducer 2602. For example, for an IVUS transducer 2602 designed to image predominately in the radial direction, it may be desirable to align the IVUS transducer 2602 with the distal end of the loop elements 2608 as shown in FIG. 26A. Alternatively, if the IVUS transducer 2602 is configured to image in a more forward looking direction, i.e. FLIVUS, it may be desirable to place the IVUS transducer 2602 proximally of the distal end of the loop elements 2608, as shown in FIG. 26B.

In some embodiments, as illustrated in FIG. 26D, the IVUS transducer 2602 can be located on the distal portion of the retrieval sheath 2610. In some embodiments, the IVUS transducer can be located proximally of the flexible, invertable tip portion 2612 of the retrieval sheath 2610. In other embodiments, the IVUS transducer 2602 can be located at the distal tip in place of the flexible, invertable tip portion 2612.

In some embodiments, as illustrated in FIGS. 26E and 26F, the IVUS transducer 2602 can be located on the shaft 2606 of the snare 2604. The IVUS transducer 2602 can be located on the distal end of the shaft 2606 around the connection between the loop elements 2608 and the shaft 2606, as shown in FIG. 26E. In some embodiments, the IVUS transducer 2602 can be located on the distal portion of the shaft 2606 proximally of the connection 2614 between the loop elements 2608 and the shaft 2606, as shown in FIG. 26F.

In some embodiments, as illustrated in FIG. 26G, the IVUS transducer 2602 can be located on the distal end of a guide catheter 2620 in which the retrieval system 2600 can be inserted through. A guidewire 2630, with an optional pressure sensor 2632, can be used in conjunction with the guide catheter 2620 and IVUS transducer 2602 to navigate through the vasculature to the deployed filter or device.

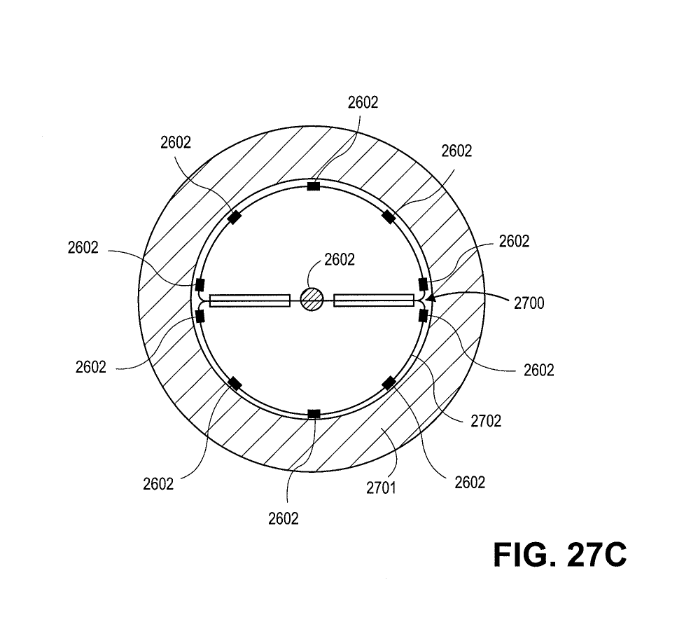

In some embodiments, as illustrated in FIGS. 27A-27C, the loop elements of the snare can function as a centering device 2700 that positions the IVUS transducer 2602 in the central portion of the lumen of the vessel 2701. In some embodiments, keeping the IVUS transducer 2602 centered within the lumen of the vessel 2701 maintains or enhances the imaging quality of the IVUS transducer 2602. The centering device 2700 can have two or more loop elements 2702 that extend radially outwards from the catheter or elongate member 2704 that carries the IVUS transducer 2602. For example, the centering device 2700 can have 2, 3, 4, 5, 6, 7, or 8 loop elements 2702. The loop elements 2702 can be attached to the catheter or elongate member 2704 in various locations and configurations as described above for the attachment of the snare loop elements 2608 to the snare shaft 2606. In some embodiments, the loop elements 2702 extend radially outwards with little axial extension. In other embodiments, the loop elements 2702 extend radially outwards and also axially in a distal and/or proximal direction. In some embodiments, a sheath 2706 can be used to cover the loop elements 2702 when the centering device 2700 is in a stowed configuration. The sheath 2706 can be retracted or the elongate member 2704 can be advanced relative to the sheath 2706 in order to deploy the loop elements 2704 in a deployed configuration. In some embodiments, the degree or amount of radial deployment of the loop elements 2702 can be controlled be controlling the amount the sheath 2706 is retracted or the elongate member 2704 is advanced. Therefore, for example, in a smaller vessel, the sheath 2706 can be retracted to a lesser amount than in a larger vessel, thereby resulting in radial deployment of the loop elements 2706 to an appropriate degree suitable for the smaller vessel.

As illustrated in FIG. 27C, the loop elements 2608 can additionally or alternatively be used to position an array of IVUS transducers 2602 around the periphery of the lumen and along or proximate the lumen wall. In some embodiments, the IVUS transducers 2602 can be integrated into wire based loop elements 2608 to form the array. The IVUS transducers can be placed on the distal portions of the loop element that is configured to abut against the lumen wall. In some embodiments, the IVUS transducers can be spaced evenly around the lumen wall when deployed. This array of IVUS transducers can be used to generate a sharp image of the tissue/lumen interface, along with any objects located within or near the tissue/lumen interface, such as a retrieval feature of a device that is located against or proximate the lumen wall.

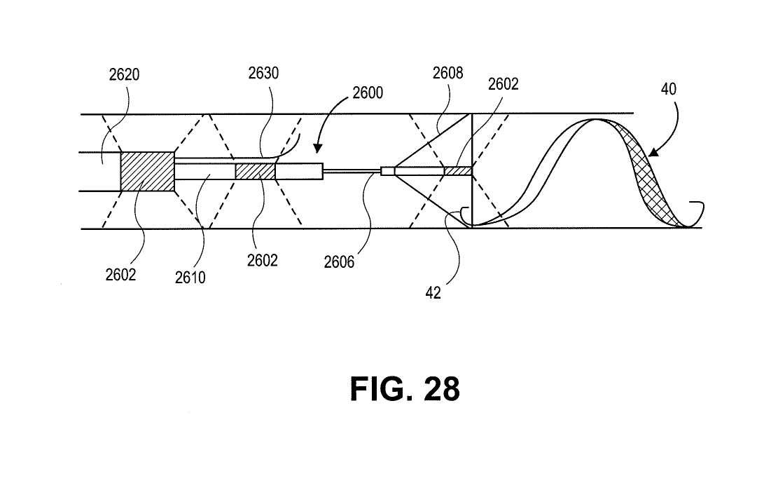

FIG. 28 illustrates a method of using a retrieval system 2600 having one or more IVUS transducers 2602 to retrieve a filter 40 from a body lumen. IVUS transducers 2602 can be located on the snare shaft 2606, the retrieval sheath 2610 and/or the guide catheter 2620, as described above. For example, a guidewire 2630 and the guide catheter 2620 can be inserted into the vessel through the a peripheral vessel, such as the femoral vein, for example, and navigated using IVUS imaging and/or fluoroscopy to the filter 40 location in, for example, the inferior vena cava. The retrieval device 2600 can be inserted through the guide catheter 2620 and IVUS imaging using any one of the IVUS transducers 2602 can be used to determine the location and orientation of the retrieval feature 42 on the filter. For example, the IVUS transducer 2602 on the distal end of the shaft 2602 can be used to align the distal end of the loop elements 2608 with the retrieval feature 42 of the filter 40, ensuring proper capture of the retrieval feature 42 with the retrieval device. If needed, the loop elements 2608 can be rotated to effect capture of the retrieval feature 42.

In some embodiments, the echogenicity of the loop elements 2608 can be increased by employing twists or braids of two or more wires to form the loops. In some embodiments, an echogenic material can be used to coat the loop elements 2608 and other parts of the snare. For example, various echogenic features as described below can be incorporated into the loop elements 2608 and any other feature of the retrieval system 2600. In addition, echogenic materials and features can be incorporated into the filter device, as described below, in order to enhance its retrievability under IVUS imaging.

Filters are more complex structures in contrast to the relatively simple designs found in catheters and needles. In a more complex device like a filter there is a need to identify specific portions within the device during some medical procedures. In addition, it would be advantageous as well to determine the orientation of the device including components within the device to one another (as used for determining deployment, retrieval and the various intermediate stages thereof) as well as the overall filter orientation to the surrounding lumen or vessel. In contrast to the conventional techniques using location of the tip or start or end of the entire length, a more complex structure such as a filter position, orientation or relative placement information would yield specific benefits. In some cases, aspects, portions or attributes of the overall filter or filter components or portions will enable more useful determinations about the filter in relation to the physiological environment. In one aspect, an intravascular ultrasound (IVUS) catheter and processing system or signal processing algorithm is used to confirm filter sizing selection, guidance for filter placement, filter implantation steps, filter and/or vessel measuring using IVUS before during and/or after steps to confirm sizing selection and fit is appropriate under the physiologic environment and for confirmation and/or documentation of proper sizing selection, placement, engagement or degree of engagement of fixation elements (if present), clot burden, orientation and/or deployment in a patient or physician medical record.

In one aspect, embodiments of the present invention are directed toward medical devices having a complex shape or that are configured to move from stowed to deployed configurations that may also have specific orientation and placement criteria for proper use in a lumen, vessel or hollow organ. One such complex device is an IVC filter. Aspects of the present invention include such devices employed within the human body which have enhanced ultrasound visibility by virtue of incorporation of an echogenic material using any of the techniques described herein alone or in any combination.

In one aspect, there are described herein various alternative filter designs for increasing the echogenicity of the filter. A filter with enhanced echogenic characteristics may include one or more than one of: (a) a modification to one or more components of the filter to enhance the echogenic characteristics of the component; (b) formation of dimples into a component surface of sufficient number and scaled to a suitable size, shape, orientation and pattern for use with intravascular ultrasound systems; (c) protrusions formed in, placed on or joined to a filter surface; (d) roughening one or more surfaces of a filter, for example using a chemical process, a laser or bead blasting technique; and (e) altering one or more steps of a filter manufacturing technique to introduce cavities, voids or pockets to locally modify or adapt one or more acoustic reflection characteristics to improve echogenicity in one or more specific regions of a filter. One example of the manufacturing alteration is to introduce gaps between the segments of tubing or coverings whereby the gap provides the echogenic enhancement. In addition, cavities, voids, pockets, dimples, gaps and the like may be left empty or, optionally, filed, partially filed or lined with any of the echogenic materials described herein.

In one aspect, there are provided embodiments of a filter having enhanced echogenic characteristics in or related to at least one or a portion of: an proximal end, a distal end, a terminal proximal end, a terminal distal end, a retrieval feature, an atraumatic tip on a retrieval feature, a mid-strut region, a leg or strut portion having at least one orientation attribute to another portion of the filter, an indicia of a location of a fixation element or a retrieval feature, a location on a portion of the filter selected such that in use with a particular fixation element the marker in a location that indicates that the fixation element is fully deployed into a wall of a lumen or portion of a vessel or hollow organ (i.e., the marker is against the lumen wall or nearly so when the fixation element is fully engaged. As such, see the marker against the wall indicates proper deployment, spaced from or not visible would indicate, respectively, not fully engaged or over penetration); a portion of the distal tip and/or an elongated portion. The above described methods may also be applied to the other techniques and alternatives described herein.

In still further embodiments, a portion, component or aspect of an intraluminal filter may have enhanced echogenic attributes by applying a coating or sleeve containing one or more of the echogenic materials disclosed herein or fabricated according to any of the techniques or having any of the attributes to enhance echogenic qualities as described herein. In some aspects, the enhanced echogenic attributes are provided by the incorporation into, application onto or within a component or portion of a filter one or more echogenic materials or echogenic markers in a specific configuration, location, orientation or pattern on the filter.

Enhanced echogenic markers or locations may be devised and placed for use individually or in combinations such as to facilitate the identification to an IVUS system or ultrasound imaging modality an indication or signature for a specific location on a filter, such as, for example, a retrieval feature, a terminal proximal end, a terminal distal end, a location of a fixation element or a location of some other indicia that identifies a specific aspect of a particular filter design. In addition or alternatively, two or more enhanced echogenic markers or portions may be used in combination to provide additional information about a filter such as orientation with in a vessel, confirmation of deployment or a portion of a deployment sequence, confirmation of final placement, confirmation of migration or lack of migration, confirmation of retrieval or progress in a retrieval sequence and the like according to the various processes and used for filters within the vasculature or in lumens of the body. In another specific embodiment, the use of IVUS techniques with embodiment of the echogenic enhanced filters describe herein may also be used to measure the diameter of the vessel at specific device locations indicated by the echogenic markers during or after deployment or retrieval of a filter.

In still further aspects, the use of IVUS techniques with embodiment of the echogenic enhanced filters describe herein may also be used to determine, detect or indicate inadequate dilation, adequate dilation, filter expansion, degree of filter expansion, filter—vessel engagement and degree or engagement, strut/leg/anchor position and other attributes relating to the interaction between the filter and the surrounding physiological environment.

Still further, the echogenic markers are positioned with regard to the likely or planned positioning of the IVUS transducer and/or likely pathways for acoustic energy used by the imaging system. By way of example, if the IVUS transducer is forward looking, then those forward looking aspects of the filter will be provided with the enhanced echogenic aspects. In another example, if the IVUS transducer is cylindrically shaped and will be positioned through the interior portion of a filter then the filter will be provided with enhanced echogenic aspects on interior surfaces or portions that would receive acoustic energy from such as transducer in such a position. Other modifications are within the scope of the invention based on the particular style of IVUS transducer used, the position relative to the filter and the placement and type of echogenic feature incorporated into the filter. Put another way, the echogenic enhancements of the filters described herein are selected, designed and positioned on the filter with regard to the IVUS sensor type, acquisition mode and position relative to the filter. Additional details in the use of IVUS with filters is further described in U.S. Pat. Nos. 6,645,152 and 6,440,077, both of which are incorporated herein by reference in their entirety for all purposes.

In one aspect, the placement and signature of such enhanced echogenic markers are discernible to a human user viewing an ultrasound output alone or in combination with being discernible to a computer system configured for the processing of an ultrasound return including a return from the enhanced echogenic filter. Additional aspects of the formation and use of echogenic materials is made with reference to the following US patents and patent Publications, each of which is incorporated herein by reference in its entirety: US 2010/0130963; US 2004/0230119; U.S. Pat. Nos. 5,327,891; 5,921,933; 5,081,997; 5,289,831; 5,201,314; 4,276,885; 4,572,203; 4,718,433; 4,442,843; 4,401,124; 4,265,251; 4,466,442; and 4,718,433.

In various alternatives, the echogenic material may either be applied to a portion of or a component of a filter in any of a number of different techniques.

In one example, an echogenic component or additive is applied to or incorporated into a filter or portion of a filter as a selective coating applied to a portion or component of a filter.

In one example, an echogenic component or additive is applied to or incorporated into a filter or portion of a filter as a mold formed to be placed over or joined to a portion of component of a filter.

In one example, an echogenic component or additive is applied to or incorporated into a filter or portion of a filter as an extruded sleeve formed in a continuous segment to cover a portion or component of a filter. In one embodiment, one of the inner tubular member or the outer sleeve or coating may be fabricated of a material according to the present invention, having increased echogenicity, with the other of the inner tubular member fabricated of a biocompatible polymer such as polyurethane or silicone rubber, for example.

In one example, an echogenic component or additive is applied to or incorporated into a filter or portion of a filter as a compound or two layer structure comprising an inner tube and an outer tube or sleeve with one or both of the tubes made from or including or incorporating one or more echogenic materials or modifications as described herein. In addition or alternatively one or both sleeves, tubes described herein may include or encapsulate an echogenic marker or component of specific shape or geometry, for example, as in the case of a tube structure having within the sidewall of the tubing a coiled structure. In one aspect, the coiled structure is made from an echogenic material and the windings are provided in a manner that is useful in any of the aspects of the filter described herein. The coil may have a particular size or variation in size, pitch or variation in pitch or other attribute useful in providing an echo identifiable aspect of the filter property being determined. In one specific embodiment, the dimensions of the coil or other echogenic material has dimensions selected for increasing acoustic reflection with regard to the resolution or processing algorithms used in the imaging ultrasound system.

In one example, an echogenic component or additive is applied to or incorporated into a filter or portion of a filter as a braided structure incorporated into a compound or two layer structure comprising an inner tube and an outer tube or sleeve with one or both of the tubes made from or including or incorporating one or more braid comprising echogenic materials or modifications as described herein. In addition or alternatively one or both sleeves, tubes described herein may include or encapsulate an braid formed into an echogenic marker or component of specific shape or geometry, for example, as in the case of a tube structure having within the sidewall of the tubing a braided structure. In one aspect, the braided structure is made from an echogenic material and the braided is a small diameter that is when wound around the tubes or sleeve or directly onto a portion of or component of a filter. The winding pattern and spacing of the braided materials are provided in a manner that is useful in any of the aspects of the filter described herein. The braid may have a particular braid strand composition, structure, size or variation in size, pitch or variation in pitch or other attribute useful in providing an echo identifiable aspect of the filter property being determined. One or more of the strands in the braid may be formed from an echogenic material. One or more of the strands may be formed from a material having improved radiopaque characteristic. One or more of the strands may be formed from a material having both echogenic and radiopaque properties. The strands of a braid may be combined using any of the above described strand characteristics.

In another alternative, in still another example, an echogenic component or additive is applied to or incorporated into a filter or portion of a filter as the a series of short segments placed adjacent to one another along a portion or component of a filter in either a close packed or spaced arrangement. In another embodiment, the spacing or voids between adjacent segments may also be adjusted or selected so as to enhance echogenic capabilities of the filter using the material difference introduced by the spacings or voids.

In another alternative, in still another example, an echogenic component or additive is applied to or incorporated into a filter or portion of a filter as a tubing or sleeve suited to heat shrink operations. In one aspect, there is a manufacturing or assembly steps of sliding one or more sleeves over portion of the filter then apply heat to shrink down the segment about the portion of the filter. In particular, various embodiments provide for the specific placement of such a shrink fit tubing having enhanced echogenic characteristics as described herein. It is to be appreciated that the sleeves, segment or tubes may be provided from or have echogenic modifications or elements incorporated into suitable materials such as, for example, ePTFE, PTFe, PET. PVDF, PFA, FEP and other suitable polymers. Still further, these and other materials may be formed in shapes other than tubes but may also take the form of strands, lines, fibers and filaments to be applied in accordance with the echogenic enhancement techniques described herein. In some embodiments, the tubes or segments applied to a filter may have the same or different composition as well as have the same width or different widths. In one aspect, the width or thickness of a plurality of bands is used to provide a code or information about the filter. The use of echogenic bands of different widths is a marking technique similar to the way that different size and color rings on a resistor are arranged in a pattern to describe the resistor's value.

In another alternative, in still another example, an echogenic component or additive is applied to or incorporated into a filter or portion of a filter is extruded over a portion of or a component of the filter.

In another alternative, in still another example, an echogenic component or additive is applied to or incorporated into a filter or portion of a filter is by bonding an echogenic material or components to the filter using a suitable adhesive or bonding technique.

In any of the above described configurations, the portion or component of the filter may be modified with dimples, grooves, pockets, voids. In other aspects, there may be one or more full or partial circumferential recesses, rings, surface diffraction gratings or other surface features to selectively enhance or provide an echogenic property in that portion of the filter, to aid in or foster the application of the echogenic materials. In still further aspects, any of above described surface modifications may also be used to uniquely identify a portion of a filter or any of the above in any combination.

In still further aspects of any of the above echogenic markers or attributes the thickness of the sleeve or coating or component may decrease at its proximal and distal ends to provide for a smooth outer surface. As yet an additional alternative, a coating, marker or other echogenic material may extend proximally to or closely adjacent to the distal end or the distal end or both of the filter component or filtering device.

In still other alternatives or combinations, some filter design embodiments alter components of the filter to enhance echogenicity such as, for example, material selection to incorporate echogenic materials. Examples of echogenic materials include palladium, palladium-iridium or other alloys of echogenic materials.

In some embodiments, echogenic microbubbles are provided in a portion of a filter to enhance the acoustic reflections of that aspect of the filter. Echogenic microbubbles may be prepared by any convenient means and introduced into the component or portion thereof or by a coating or sleeve or shell or other transferring means or mixed with a polymer or other suitable base compound prior to extension of extrusion, molding casting or other technique. The echogenic microbubbles may be pre-prepared or prepared inside the component or element or marker as appropriate. Aspects of the preparation or use of microbubbles are described in U.S. Pat. Nos. 5,327,891; 4,265,251; 4,442,843; 4,466,442; 4,276,885; 4,572,203; 4,718,433 and 4,442.843. By way of example, echogenic microbubbles can be obtained by introducing a gas, e.g. carbon dioxide, into a viscous sugar solution at a temperature above the crystallization temperature of the sugar, followed by cooling and entrapment of the gas in the sugar crystals. Microbubbles can be formed in gelatin and introduced into a component or portion of a device. Microbubbles can also be produced by mixing a surfactant, viscous liquid and gas bubbles or gas forming compound, e.g. carbonic acid salt, under conditions where microbubbles are formed.

In still further alternatives, there is also the incorporation of dual mode materials (radiopaque and echogenic) into a polymer then used to form part of, be applied or otherwise incorporated with a filter device as described herein. Some of these polymer compounds may be fabricated to enhance aging and shelf life and have other beneficial attributes. In one aspect, a filter or portion thereof includes one or more selected segments that are constructed using visibility materials compounded with one or more polymeric materials that make the selected segments visible using both fluoroscopy and ultrasonic imaging. In one specific example, the visibility material may take the form of tungsten and/or tungsten carbide particles dispersed within a polymeric material. In one specific aspect, the radiopaque and echogenic material includes tungsten and/or tungsten carbide particles distributed within a base polymeric material.

In one embodiment, a portion of or a component of a filter includes or has been modified to have an inner layer including a radiopaque and echogenic material. In one alternative, the radiopaque and echo genic material includes particles distributed within a base polymeric material (i.e., a first polymeric material) including a polyether block amide; and an outer layer including an additional polymeric material (i.e., a second polymeric material). In certain embodiments, the additional polymeric material is a thermoplastic elastomer. Optionally, the additional polymeric material is more resistant to hydrolysis and/or oxidation than the base polymeric material.

In still further aspects, a component, a portion or an element added to a filter may be regarded as an echogenic body member that is a part of an echogenic filter to be sonically imaged. The echogenic body member is at least partially made up of a composite material which is echogenically imagable in the patient, such as by the use of ultrasonic imaging equipment used either within the patient or external to the patient. In one aspect, a composite material includes matrix material with discrete acoustic reflective particles embedded in matrix material. In one aspect, the matrix material is a biocompatible plastic. Examples of suitable plastics may include urethane, ethylene, silicone, polyethylene, tetrafluorethylene. In one aspect, a matrix is a formable, pliable material which may be molded and/or extruded to a variety of shapes, depending upon a specific application. The sound reflective particles are embedded in matrix material. Particles are, by way of example, made of a hard material, such as small glass particles that are solid or filled with an acoustically reflective medium. In one aspect, glass particles having a generally spherical shape forming glass microspheres. Glass microspheres with an outer diameter of about 5 microns is one acceptable size. Other sized particles may be utilized as, for example, ranging between 1 and 50 microns and beyond. Particles sized below the resolution size of the imaging ultrasound system in use may be arranged into patterns of sufficient size and orientation to the acoustic waves that result in a discernible feature by the imaging ultrasound system. Furthermore, the particles do not necessarily have to be spherical, or may be partially spherical. Still further, the shape of the particle could be altered to enhance acoustic reflection by presenting different shapes of particles, sizes of particles and combinations thereof to modify acoustic characteristics of the matrix material. By way of example, the particles may be shaped into an “Ordered array.” “Ordered arrays” can take the form of a macrostructure from individual parts that may be patterned or unpatterned in the form of spheres, colloids, beads, ovals, squares, rectangles, fibers, wires, rods, shells, thin films, or planar surface. In contrast, a “disordered array” lacks substantial macrostructure.

By way of example, an echogenic marker may comprise particles that individually are below the resolution of the imaging ultrasound system. The echogenic marker is the combination of these below imaging ultrasound resolution particles in combination, in 1D, 2D or 3D patterns, in graphic arrays, or in machine readable combinations to make a signature. Based on the specific characteristics of the combination of particles, the acoustic returns from an echogenic marker or combination of echogenic markers may be visually perceptible in a display for interpretation by a user or may be detected and interpreted by one or more acoustic reflection or spectral processing algorithms within a imaging ultrasound processing system.

In one aspect, the echogenic material is fabricated by incorporating nanometer sized particles of sonically reflective materials, for example iron oxide, titanium oxide or zinc oxide into a biocompatible polymer. In one method of fabrication, the acoustically reflective particles are mixed with a powdered thermoplastic or thermosetting material such as a polyether amide, a polyurethane or an epoxy, or polyvinylchloride followed by thermal processing of the mixture to provide a material of increased sonic reflectance which may be applied as a coating on medical devices of the type discussed above or may be incorporated as a structural component of the medical devices as described herein.

In still further embodiments and aspects, the particles included to provide echogenic enhancements may be selected, arranged or incorporated to provide acoustically geometrically tuned nanostructures, microstructures or macrostructures. The particles provided herein are formable in all shapes currently known or to be created for acoustic reflection enhancement. In non-limiting examples, the nano-, micro- or macro-particles are shaped as spheres, ovals, cylinders, squares, rectangles, rods, stars, tubes, pyramids, stars, prisms, triangles, branches, plates or comprised of an acoustically reflective surface or where one or more surfaces is adapted such as by roughening or dimpling or other technique used to alter acoustic reflection properties. In non-limiting examples, the particles comprise shapes and properties such as plates, solid shells, hollow shells, rods, rice shaped, spheres, fibers, wires, pyramids, prisms, or a combination thereof.

In one specific aspect, a partially spherical surface may be provided on the outside and/or the inside of particles, as for example a particle with a hollow spherical space therein. Particles are made up of a different material than the matrix. While desiring not to be bound by theory, it is believed that a spherical shape provides for sound reflections at a variety of angles regardless of the direction from which the ultrasonic sound waves are emanating from, and accordingly, are more likely to reflect at least a portion of the transmitted signal back to the ultrasonic receiver to generate an image. Since many of matrix materials available are relatively ultrasonically transparent in a patient, sound reflective particles provide adequate reflection. The use of a composite, rather than a solution, provides adequate size for acoustic reflection off of the discrete particles embedded in the matrix. As indicated, a variety of materials may be utilized for the sound reflective particles, such as aluminum, hard plastic ceramics, and, metal and/or metal alloys particles, and the like. Additionally, liquids, gases, gels, microencapsulants, and/or suspensions in the matrix may alternatively be used either alone or in combination, so long as they form a composite with the desired ultrasonically reflective characteristic.

Any of the above embodiments, alternatives or filter modifications to enhance echogenic characteristics may also be designed or implemented in such a way as to provide an echogenic identifiable or unique trait or acoustic reflection signature that may be registered by a human operator looking at a display or identified using signal processing techniques of a return containing acoustic reflections from the filter in an imaging ultrasound system. In one example, there is a surface of the filter having one or more echo registerable or identifiable feature, mark or indication in a position useful for determining one or more of: a location of an end of a filter; a location of a fixation element on a filter; a location of a retrieval feature on a filter; an orientation of one or more of a leg, a strut, a filter or an end of a filter relative to another of a leg, a strut, a filter or an end or the orientation of the overall filter to a lumen, vessel or hollow organ in a body. Moreover, in another widely applicable aspect of providing enhanced imaging characteristics to a filter as described herein, the characteristic or modification—however added or incorporated into the filter—enable a filter, a filter component or a specified portion of a filter to be more readily imaged by intravascular ultrasound as described herein. In still another aspect, the characteristics or modification to the filter are oriented and positioned in order to facilitate IVUS imaging via an IVUS probe borne by a filter deployment or retrieval catheter, snare, or other implement provided to facilitate the use of intravascular filters.

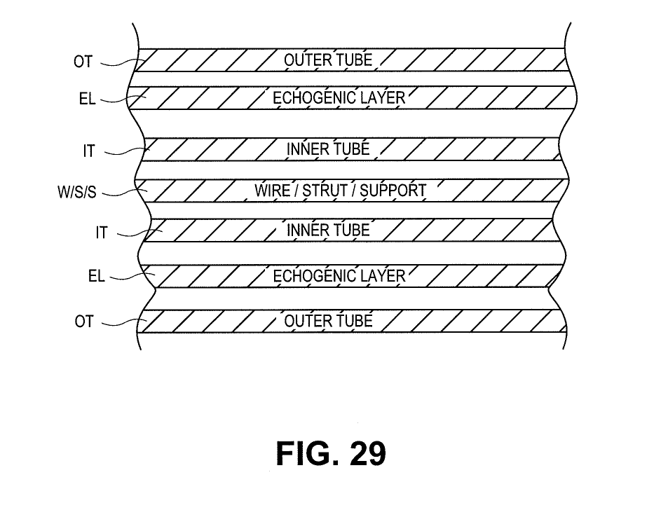

FIG. 29 is a section view of a wire strut or support element of a filter (w/s/s) having multiple segments in a concentric arrangement. In this illustrative embodiment, the wire is encased in alternating tube segments. There is an inner tube (IT) directly adjacent to the wire. There is an echogenic segment layer (EL) adjacent to the inner layer. The inner tube may be selected to act as bonding layer to increase adhesion between the echogenic layer and the filter wire, strut or support member. In this embodiment, there is an outer tube (OT) over the echogenic layer. In alternative configurations, either or both of the inner tube or the outer tube may be omitted. The echogenic layer is a segment having one or more of the echogenic characteristics described herein.

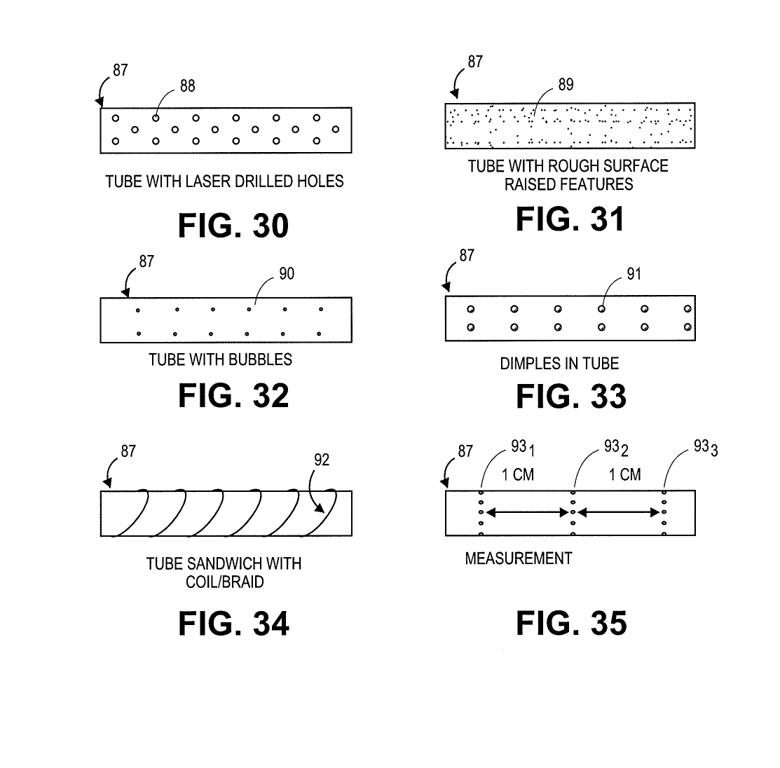

FIGS. 30-35 provide various exemplary embodiments of a segment 87 having one or a plurality of one or more than one type of echogenic characteristic, property or feature added thereto. Each of the illustrated echogenic adaptations applied to segment 87 along with segment 87 itself may be sized, scaled and/or shaped as described herein as needed based upon the requirements of the portion of the filter and the echogenic characteristic.

FIG. 30 is an embodiment of a segment 87 having one or a plurality of laser drilled holes 88 formed therein. The diameter and the shape of the holes may be selected based upon the size of the filter or filter component to which the segment 87 will be attached. The holes 88 may be completely through the wall of the segment or only partially through the wall. The holes 88 may be formed in any pattern, spacing or orientation as described herein.

FIG. 31 is an embodiment of a segment 87 having one or a plurality of raised features or alternatively roughed portions 89 formed thereon. The size and shape of the raised features or the roughness of the surface may be selected based upon the size of the filter or filter component to which the segment 87 will be attached. The raised features or portions of roughness 89 may be formed in any pattern, spacing or orientation as described herein.

FIG. 32 is an embodiment of a segment 87 having one or a plurality of bubbles 90 formed therein. The size, shape, pattern, and manner of incorporating one bubble 90 or a plurality of bubbles 90 into the segment 87 may be selected based upon the size of the filter or filter component to which the segment 87 will be attached. The bubbles 90 may be formed within the segment sidewall, near the surface of the segment sidewall or near the inner surface of the sidewall. The bubble or bubbles 90 may be formed in any pattern, spacing or orientation as described herein.

FIG. 33 is an embodiment of a segment 87 having one or a plurality of dimples 91 formed therein. The diameter and the shape of the dimples may be selected based upon the size of the filter or filter component to which the segment 87 will be attached. The dimples 91 may be formed in any pattern, spacing or orientation as described herein.

FIG. 34 is an embodiment of a segment 87 having a coil or braided structure 92 within or about the segment 87. The size, shape, pattern, and manner of incorporating the coil or braid 92 into the segment 87 may be selected based upon the size of the filter or filter component to which the segment 87 will be attached. The coil or braid 92 may be formed within the segment sidewall, near the surface of the segment sidewall or near the inner surface of the sidewall. The coil or braid 92 may be part of a sandwich structure as illustrated and described in FIG. 29. The coil or braid 92 may be formed in any pattern, spacing or orientation as described herein to enhance the echogenic characteristics of the filter or filter portion attached to the segment 87. The coil or braid 92 may be continuous along the entire length of a segment 87 or, alternatively, the coil or braid 92 may be in short lengths selected so that a plurality of coils or braids are provided within a single segment 87.

FIG. 35 is an embodiment of a segment 87 having a plurality of echogenic markers 93 arrayed in rings 93.1, 93.2 and 93.3. For purposes of illustration the rings are shown in an orientation that is generally orthogonal to the central longitudinal axis of the segment 87. The rings are shown with a sample spacing of 1 cm between them. The spacing may be any suitable distance based on the factors described herein such as filter size and physiological environment. Similarly, the rings may be angled in other orientations relative to the longitudinal axis of the segment. For example, some ring may be in one angular orientation while other rings may be in a different angular orientation where the angular orientation or patent of orientation is utilized to provide one or more of the filter functionality or echogenic characteristics described herein. In some specific configurations, the spacing and sizes used are in the millimeter range. In some specific configurations, the spacing and sizes are in the micron range. In some specific configurations, the size and/or spacing of a ring or between adjacent rings are in a combination of mm and micron ranges for sizes, spacings and features. The size and spacing of the echogenic markers 93 may be selected based upon the size of the filter or filter component to which the segment 87 will be attached. The markers 93 may be formed in any pattern, spacing or orientation as described herein in order to facilitate a measurement using the markers. Still further, the markers 93.1, 93.2 and 93.3 may be utilized for provide for other filter characteristics as described herein.

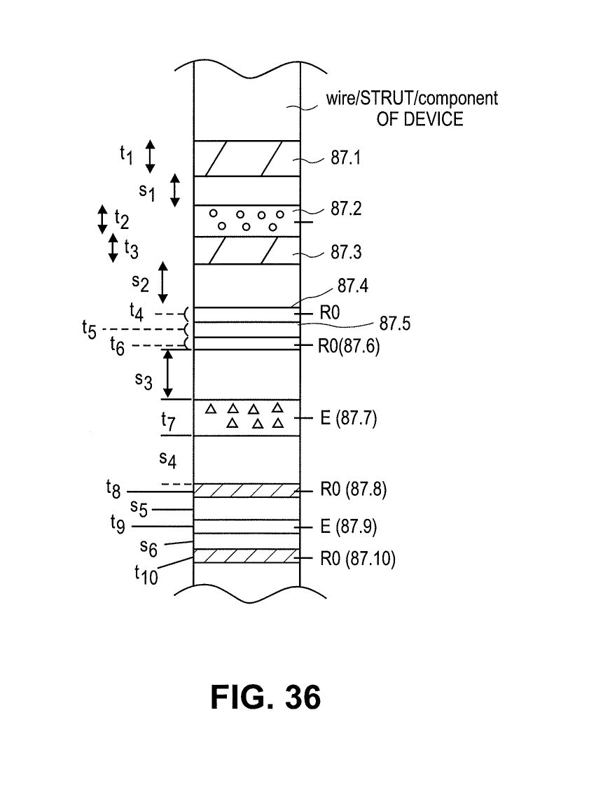

FIG. 36 illustrates various alternative configurations for a segment used alone or in conjunction with other segments. The segments are illustrated along an exemplary wire, strut, or component of a filtering device. The segments may have different characteristics to enable the segment to be more readily imaged by a medical imaging modality used externally, internally or intraluminally. In one aspect, the segment characteristics are selected to provide for imaging enhancements for a filter being used within a vein or an artery. In another aspect, the segments may have different characteristics to enable the segment to be readily imaged by intravascular ultrasound as described herein. In still another aspect, the segments are oriented and positioned in order to facilitate IVUS imaging via an IVUS probe borne by a filter deployment or retrieval catheter, snare, or other implement. In one illustrative embodiment, the segments are selected and arrayed to facilitate imaging utilizing IVUS and an external medical imaging modality. In one exemplary embodiment, the external imaging modality is x-ray.

Also illustrated in FIG. 36 is the use of a combination of different echogenic characteristics (designated E) and radio-opaque characteristics (designated RO). These characteristics may be any of those described herein in any combination. The echogenic characteristic of a segment may be the same as another segment in a grouping such as in the E segments 87.9 and 87.5. Alternatively, the echogenic characteristic of a segment may be different from those in an adjacent group as with segments 87.2, 87.5 and 87.7.

FIG. 36 also illustrates not only that different characteristic and properties of segments may be used but also how variable segment dimensions may be used to aid in echogenic enhancement of a filter. As illustrated, the segments have different widths or thicknesses as indicated along the longitudinal axis of the wire, strut or component. As such, FIG. 36 illustrates a series of imagine enhancing segments 87.1-87.10 having a variety of width or thickness values t1-t10. In one embodiment, the segments are configured as short rings or bands. The thickness of segments in groups may be similar as illustrated in segments 87.1, 87.2 and 87.3 where the thicknesses t1, t2 and t3 are about the same. Similarly, segments 87.4, 87.5 and 87.6 illustrate segments of similar width or thickness where t4, t5 and t6 are about the same value. Similarly, segments 87.8, 87.9 and 87.10 illustrate segments of similar width or thickness where t8, t9 and t10 are about the same value.

FIG. 36 also illustrates how segments within a group or groups of segments may have a variety of different spacing (s1-s6) to provide enhancements to a filter for improving medical imaging modality characteristics. For example, in the segment grouping of 87.1, 87.2 and 87.3, there is a spacing s1 between segment 87.1 and segment 87.2 but then no spacing between segments 87.2 and 87.3. A spacing s2 is shown between segment 87.3 but then no spacing in the combination segment grouping formed by segments 87.4, 87.5 and 87.6. A spacing of s3 is shown between the three segment combination of 87.4, 87.5 and 87.6 to the single segment 87.7. The single segment 87.7 is spaced apart by spacing s4 from the equally sized (i.e., t8=t9=t10) and equally spaced (i.e., s5=s6) group of segments 87.8, 87.9 and 87.10. It is to be appreciated that in various alternative embodiments, the spacing used in groups of segments or between groups of segments may be the same or variable.

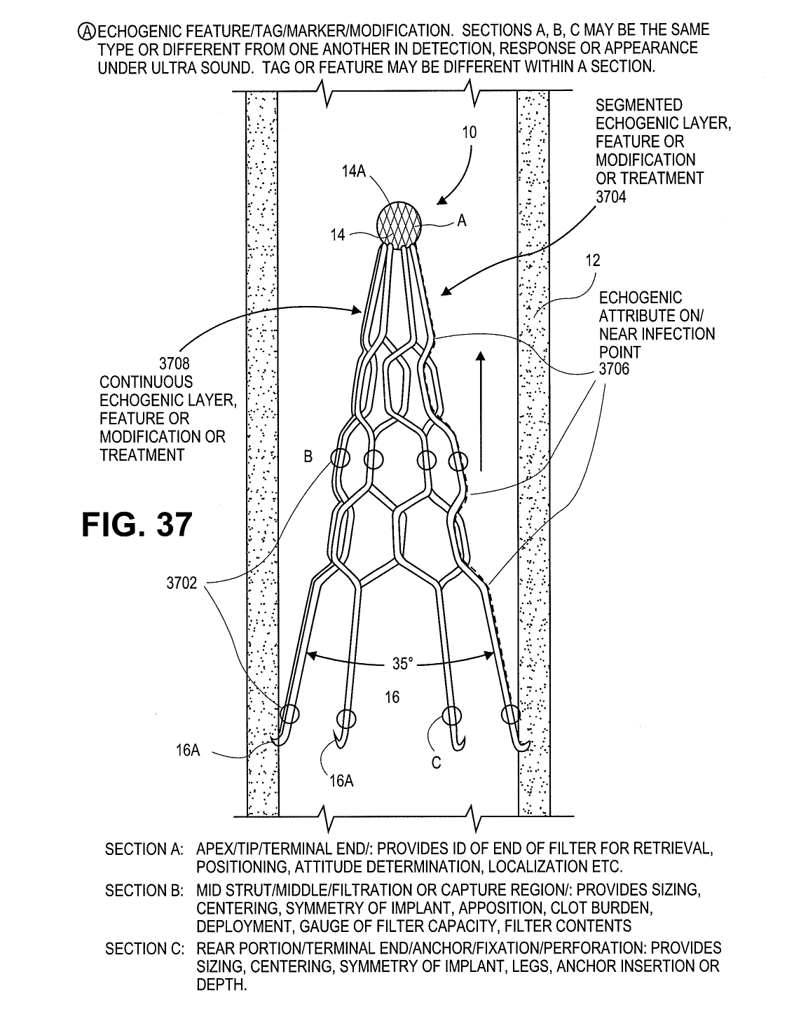

FIG. 37 is a view of an exemplary filter illustrating various alternative aspects of providing a filter with improved echogenic characteristics. The filter illustrated is a conical filter. It is to be appreciated that the filter of FIG. 37 is merely representative of one type of filter. It is to be appreciated that the various alternative enhancement, modifications and treatments described herein may be provided to any intravascular or intraluminal filter. The exemplary filter is dividing into three general sections A, B and C. Sections A. B and C may be the same type of enhancement or have an enhancement different from one another section. In addition, the type of enhancement in each section may be the same or different from one another in detection, response or appearance under ultrasound. In addition, a tag, feature or enhancement may be different within a section. Circles 3702 are used to indicate exemplary locations for an echogenic feature, tag, marker or modification to an enhanced filter 10. The illustrative embodiment in FIG. 37 also illustrates a continuous echogenic layer, feature or modification or treatment 3708. The illustrative embodiment in FIG. 37 also illustrates an echogenic attribute on/near an inflection point 3706 in an enhanced filter structure 10. The illustrative embodiment in FIG. 37 also illustrates a segmented echogenic layer, feature or modification or treatment 3704 on an enhanced filter structure 10. Section A is considered the apex, tip, distal portion or terminal end depending upon filter configuration. Section B is considered the mid-strut, middle, filtration portion, debris capture portion, or thrombus collection or lysing portion depending upon specific filter configuration. Section C is considered the rear portion, proximal portion, proximal terminal portion, anchor, fixation or perforation portion depending upon a specific filter configuration. It is to be appreciated as well that the echogenic features, tags, markers or modifications illustrated for sections A, B and/or C may be of the same type or different types depending upon the echogenic signature or attribute intended for that section, group or sections or filter. As such, the echogenic features, tags, markers or modifications for a particular section may be selected from any of the various alternatives described herein.

Echogenic characteristics may be added to each of the sections based on the type of function being measured or characterized. For example, echogenic markers, features or tags may be added to Section A in order to provide, for example: identification of the terminal end, end portion or retrieval portion of a filter. Echogenic characteristics of Section A may also be used for determinations related to Section A specifically or the filter generally of filter position, positioning, attitude within the lumen, localization of the filter within the vasculature and other traits common to the characterization of intravascular devices. For example, echogenic markers, features or tags may be added to Section B in order to provide, for example: identification of the mid strut portion, middle or capture region. Echogenic characteristics of Section B may also be used for determinations related to Section B such as for sizing, centering, symmetry of implantation, placement, apposition of implant to vessel walls, clot burden, deployment status or completion, gauge of filter capacity and/or filter contents as well as filter position, positioning, attitude within the lumen, localization of the filter within the vasculature and other traits common to the characterization of intravascular devices. For example, echogenic markers, features or tags may be added to Section C in order to provide, for example: identification of the rear portion, terminal end, retrieval feature, anchor location or depth of insertion, perforation indication or other aspects of the rear or proximal portion of a filter. Echogenic characteristics of Section C may also be used for determinations related to Section C such as for sizing, centering, symmetry of implantation or placement of legs struts and the like, as well as for determination of wall apposition, anchor penetration or perforation. Still further, the markers or tags may be added to aid in determining or evaluating filter position, positioning, attitude within the lumen, localization of the filter within the vasculature and other traits common to the characterization of intravascular devices.

A filter having enhanced echogenic properties is illustrated in FIG. 37 as it appears when it is in operative position within the vasculature. In one specific aspect the filter is in use in a large blood vessel. One exemplary vessel is the vena cava. Still further, a modified filter may be employed in a different vein or even an artery. The filter is designated generally by reference numeral 10, and the wall of the blood vessel in which it is located is designated by reference numeral 12. The filter 10 includes an apical hub 14 of overall egg-shaped or tear drop configuration and which has a generally hemispherically shaped end portion 14a.

The filter 10 includes a plurality of elongated legs 16 which are of equal length and are identically configured to each other. The legs 16 are collectively arrayed in a conical geometric configuration so that the legs converge to the apical hub 14, and are symmetrically spaced about a central axis extending through the hub. Each of the legs is of equal diameter over its entire length and is made of a relatively resilient material, such as tempered stainless steel wire or the like. In addition to the echogenic attributes described herein, the legs may be coated with a polymeric, synthetic resin material having anti-thrombogenic properties. FIG. 37 illustrates an echogenic marker at the tip 14. Exemplary continuous echogenic layers, features or modifications are also illustrated along one or more legs of the filter. In addition, FIG. 37 illustrates the use of echogenic tags, features or markers at, along or near inflection points in a filter element or component. In addition, FIG. 37 illustrates to application of echogenic markers, tags or features near the fixation elements of the filter.

In still other alternative embodiments, there is provided a material capture structure having one or more echogenic enhancements alone or in combination with radiopaque enhancements. In one aspect, the filter structure used in a filter includes both echogenic and radio opaque enhancements.

An one aspect, the filter includes material capture structure in the IVC filter will be viewable under fluoroscopic and ultrasound imaging modalities, including appropriate echogenic characteristics to enhance the view of the status or condition of the material capture structure while using IVUS. Enabling the material capture structure to be viewed will allow the physician to appropriately center and verify placement of a filter.

In one aspect, the filter elements or structures are doped to incorporate one or more of echogenic or radio opaque materials or treatments. In one aspect, the membrane, filaments or strands or other structures used to form the filter structure or webbing of the filter includes a radiopaque material having high echogenic properties, such as tungsten or gold, but not limited to either.

In other embodiments, one or more membranes, filaments or portions of a filament within a material capture structure includes one or more non-metallic echogenic features, such as those described elsewhere in this specification. For example, a membrane or filament or portion thereof may include air pockets either added to the material or by the use of materials with entrained air or gas that are used. Another example may include a membrane with a plurality of holes. In one embodiment, an ePTFE suture has echogenic properties due to air content of the ePTFE material. In other aspects, a suture material or a filament or polymer strand may also include dimpled/roughened/matrix/sponge materials, additives, or modifications to provide or enhance the overall echogenic nature of the suture, filament, material or material capture structure, in whole or in part.

In one aspect, these additional materials may assist the physician in centering or placing a filter within a vessel. In another aspect, this improvement is used in conjunction with IVUS will enable the adequate viewing of the filter portion of the filter and will allow for co-registration of filter placement along with an accurate entry/removal of the catheter through the webbing of the filter.

The advantages of this inventive aspect of a filter include, for example and not limitation, filter placement, accurate representation of filter location, ease of introducing/retracting catheter, more viewable space for more accurate assessments, ability to co-register filter location with IVUS and/or ability to better place filter in desired location.

Still other aspects of the use of the innovative filter include, for example, deployment of filters, positioning of filters, sizing of filters, and estimated treatment lengths as well as suture and/or material capture structure visibility. In still other aspects of the use of the innovative filter include, for example, deployment of a vena cava filter, positioning of an IVC filter, sizing of an IVC filter, and estimated treatment lengths as well as enhanced suture visibility.

In one embodiment, there is an IVC filter delivery system with an enclosed IVC filter. This filter would have a mesh, suture, web or other material capture structure suited to the anticipated filter use. The mesh, suture, web or other material capture structure has one or more components that is doped with a highly radiopaque material for better visibility under flouro and good echogenicity for better viewing under IVUS guidance. In still further alternative embodiments, the techniques described above may be applied to one or more material capture structure described in U.S. Patent Application Publication US 2008/0147111 entitled “Endoluminal Filter with Fixation” filed Jun. 4, 2008 as U.S. patent application Ser. No. 11/969,827, (the “'7111 publication”) incorporated herein by reference in its entirety for all purposes. In one particular aspect, the filament/strand/suture 461 shown in FIG. 58 of the '7111 publication may be coated or doped as described above alone or in combination with the illustrated pharmacological coating 466.

In some embodiments, the snare handle portion can include snare deployment indicators, such as detents, that allow the operator to easily identify and achieve the different stages of snare deployment described above. For example, the operator can deploy the snare using the snare handle until the snare handle reaches a first indicator, which signifies that the snare is deployed in the first deployment stage. The operator can then further deploy the snare using the snare handle until the snare handle reaches a second indicator, which signifies that the snare is deployed in the second or intermediate deployment stage. Then the operator can further deploy the snare using the snare handle until the snare handle reaches a third indicator, which signifies that the snare is fully deployed. In some embodiments, there is a snare deployment indicator for each stage of snare deployment. In some embodiments, the loop elements of the snare have different configurations in each of the different deployment stages as, for example, described above. For example, deployment indicators can be provided to allow the operator to deploy the snare in stages as described above with respect to FIGS. 1D-1G and FIGS. 1N-1Q. As described above, a deployment stage corresponding to loop elements having an axial configuration can be particularly suited for retrieval of guidewires, leads, and other objects that are positioned transversely with respect to the snare axis. The fully deployed configuration can be particularly suitable for devices that have been designed for retrieval with the snare, such that markers can be used to align the snare with the object to be retrieved. In addition, the fully deployed configuration is particularly suitable for retrieving objects that are located near or proximate the lumen wall.

While described in various embodiments for retrieval of filters and other medical devices and objects, the sheath and snare designs may also be used to retrieve other filter devices, other embolic protection devices, and other objects. For example, filter devices and other devices described in commonly assigned, and concurrently filed U.S. Provisional Patent Application Ser. No. 61/586,661 (Attorney Docket Number 10253-701.102) is incorporated herein by reference in its entirety and for all purposes.

It is understood that this disclosure, in many respects, is only illustrative of the numerous alternative filtering device embodiments of the present invention. Changes may be made in the details, particularly in matters of shape, size, material and arrangement of various filtering device components without exceeding the scope of the various embodiments of the invention. Those skilled in the art will appreciate that the exemplary embodiments and descriptions thereof are merely illustrative of the invention as a whole. While several principles of the invention are made clear in the exemplary embodiments described above, those skilled in the art will appreciate that modifications of the structure, arrangement, proportions, elements, materials and methods of use, may be utilized in the practice of the invention, and otherwise, which are particularly adapted to specific environments and operative requirements without departing from the scope of the invention. In addition, while certain features and elements have been described in connection with particular embodiments, those skilled in the art will appreciate that those features and elements can be combined with the other embodiments disclosed herein.

What is claimed is:

1. A device for retrieving an object from a lumen defined by a lumen wall, the device comprising:
a sheath configured to fit within the lumen, the sheath having a proximal end and a distal end;
a snare slidably disposed within the sheath, the snare having a shaft with a longitudinal axis, a proximal end and a distal end and a plurality of loop elements in connection with the distal end of the shaft, wherein each of the plurality of loop element has a proximal portion and a distal portion, wherein the plurality of loop elements has a collapsed configuration within the sheath and at least one deployed configuration outside the sheath, wherein the plurality of loop elements are configured to be deployed through an opening at the distal end of the sheath, wherein the at least one deployed configuration includes a fully deployed configuration in which the plurality of loop elements are deployed such that the distal portions of the loop elements are arranged in a substantially continuous, circumferential, planar and oblong configuration that is transverse to the longitudinal axis; and
an intravascular ultrasound transducer located on the distal end of the shaft.

2. The device of claim 1 wherein the sheath includes a flexible distal tip portion that is configured to invert when the object is withdrawn into the sheath.

3. The device of claim 1 wherein the plurality of loop elements in the fully deployed configuration are angled less than 90 degrees with respect to the longitudinal axis of the shaft such that the plurality of loop elements has an axial reach both proximal and distal the distal end of the shaft.

4. The device of claim 1 wherein each of the plurality of loop elements includes at least one shape memory wire and one radiopaque wire.

5. The device of claim 4 wherein the shape memory wire is made of a nickel titanium alloy and the radiopaque wire is made of platinum.

6. The device of claim 1 wherein the proximal portions of the plurality of loop elements comprise spoke portions that are secured together with a flexible sleeve.

7. The device of any one of claims 1 to 6 wherein the object is a filter having a retrieval element and a support member, and wherein the axial reach of the loop elements in the fully deployed configuration is less than the distance between the retrieval element and the support member.

8. The device of any one of claims 1 to 6 wherein the proximal portion of the sheath and the proximal portion of the shaft are connected with a snap fitting.

9. The device of any one of claims 1 to 6 further comprising an outer sheath, wherein the sheath is disposed within the outer sheath.

10. The device of claim 9 wherein the outer sheath has greater column strength than the sheath.

11. The device of any one of claims 1 to 6 wherein the loop elements have a plurality of deployment configurations, and wherein the proximal portion of the shaft includes a plurality of indicators that correspond to the plurality of deployment configurations.

12. The device of claim 11 wherein the plurality of indicators comprise a plurality of detents.

13. The device of any one of claims 1 to 6 wherein the proximal portion of the sheath includes a first tactile identifier and the proximal portion of the shaft includes a second tactile identifier, wherein the first tactile identifier is different from the second tactile identifier.

14. The device of any one of claims 1 to 6 wherein the at least one deployed configuration includes an initial deployed configuration in which the plurality of loop elements are deployed substantially axially with respect to the longitudinal axis.

15. The device of any one of claims 1 to 6 wherein the distal portions of the plurality of loop elements in the fully deployed configuration are configured to achieve complete circumferential apposition with the lumen wall.

16. The device of any one of claims 1 to 6 wherein the at least one deployed configuration includes an intermediate deployed configuration in which the plurality of loop elements are deployed substantially transversely with respect to the longitudinal axis.

17. A device for retrieving an object from a lumen, the device comprising:
a sheath configured to fit within the lumen, the sheath having a proximal end, a distal end and a radiopaque marker offset from the distal end;
a snare disposed within the sheath, the snare having a shaft with a longitudinal axis, a proximal end and a distal end and a plurality of loop elements in connection with the distal end of the shaft, wherein the plurality of loop elements has a collapsed configuration within the sheath and at least one deployed configuration outside the sheath, wherein the plurality of loop elements are configured to be deployed through an opening at the distal end of the sheath, wherein the at least one deployed configuration includes an initial deployed configuration in which the plurality of loop elements are deployed substantially transversely with respect to the longitudinal axis; and
an intravascular ultrasound transducer located at the distal end of the sheath.

18. The device of claim 17 wherein the at least one deployed configuration includes a fully deployed configuration in which the plurality of loop elements are deployed in substantially circular configuration.

19. The device of claim 17 wherein the radiopaque marker is offset about 3 to 5 mm from the distal end of the sheath.

20. The device of claim 17 wherein the at least one deployed configuration includes a fully deployed configuration in which the plurality of loop elements are deployed in substantially oblong configuration.

21. The device of any one of claims 17 to 20 wherein the plurality of loop elements each includes a loop collapse facilitator.

22. The device of any one of claims 17 to 20 wherein the plurality of loop elements are secured together with sleeves.

23. A method for capturing an object in a lumen defined by a lumen wall, the method comprising:
advancing a sheath within the lumen, the sheath having a proximal end and a distal end, until the distal end of the sheath is proximal the object;
imaging the object using an intravascular ultrasound transducer,
aligning the distal end of the sheath with the object based on the image of the object;
deploying a plurality of loop elements of a snare out of the distal end of the sheath until the loop elements achieve substantially full apposition with the circumference of the lumen wall; and
capturing a portion of the object proximate to the lumen wall with at least one of the plurality of loop elements.

24. The method of claim 23, further comprising aligning a radiopaque marker offset from the distal end of the sheath with a radiopaque feature of the object.

25. The method of claim 24, wherein the radiopaque feature of the object is a retrieval element.

26. The method of claim 23, further comprising advancing the distal end of the sheath over the captured object.

27. The method of claim 26, wherein the distal end of the sheath inverts as the sheath is advanced over the captured object.

28. A method for capturing an object in a lumen defined by a lumen wall, the method comprising:
advancing a sheath within the lumen, the sheath having a proximal end and a distal end, until the distal end of the sheath is proximal the object;
determining the position of the object within the lumen;
deploying a plurality of loop elements of a snare out of the distal end of the sheath to one of a plurality of predetermined loop element deployment configurations based on the determination of the position of the object; and
capturing a portion of the object with at least one of the plurality of loop elements.

29. The method of claim 28, wherein the plurality of loop elements are deployed to the predetermined loop element deployment configuration using a deployment indicator.

30. The method of claim 28, further comprising advancing an inner sheath disposed with the sheath over a portion of the object and advancing the sheath over the entire object.
